(19) United States
(12) Patent Application Publication
(10) Pub. No.: US 2008/0014584 A1
(43) Pub. Date: Jan. 17, 2008

(54) MODULATING ANDROGEN RECEPTOR ACTIVITY

(75) Inventors: Donald J. Tindall, Rochester, MN (US); Scott M. Dehm, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/756,715

(22) Filed: Jun. 1, 2007

Related U.S. Application Data
(60) Provisional application No. 60/810,450, filed on Jun. 1, 2006.

(51) Int. Cl. C12Q 1/68; C07H 21/04; G01N 33/68

(52) U.S. Cl. 435/6; 436/86; 536/23.5

(57) ABSTRACT

This document relates to methods and materials involved in modulating (e.g., inhibiting or stimulating) the activity of an androgen receptor polypeptide. For example, this document provides materials and methods involved in identifying molecules that interact with and inhibit the activity of an androgen receptor polypeptide in androgen-independent prostate cancer cells.

### CROSS-REFERENCE TO RELATED APPLICATIONS

This application claims the benefit of U.S. Provisional Application Ser. No. 60/810,450, filed Jun. 1, 2006.

### STATEMENT AS TO FEDERALLY SPONSORED RESEARCH

This invention was made with government support under grants DK065236, DK060920, CA091956, and CA121277 awarded by National Institutes of Health. The government has certain rights in the invention.

### BACKGROUND

1. Technical Field

This document relates to methods and materials involved in modulating (e.g., inhibiting or stimulating) the activity of an androgen receptor polypeptide. For example, this document provides materials and methods involved in identifying molecules that interact with the androgen receptor polypeptide and inhibit the activity of the androgen receptor polypeptide in cells such as androgen-independent prostate cancer cells.

2. Background Information

Prostate cancer is the most frequently diagnosed male cancer and the second leading cause of cancer deaths in industrialized countries (Jemal et al., Cancer J Clin., 55:10-30 (2005)). Androgens support development and survival of normal prostate cells and prostate cancer cells by binding to and transcriptionally activating the androgen receptor (AR), a member of the nuclear transcription factor superfamily (Heinlein and Chang, Endocr Rev., 25:276-308 (2004)). The treatment regimen for locally advanced or metastatic prostate cancer aims to inhibit AR transcriptional activity through physical or chemical castration or administration of AR antagonists (Scherr et al., Urology, 61:14-24 (2003)). This approach results in reduced expression of AR target genes, such as prostate specific antigen (PSA), and concomitant tumor regression (Feldman and Feldman, Nature Reviews Cancer, 1:34-45 (2001)). However, prostate cancer invariably relapses in a form that is resistant to these hormonal manipulations, and further treatment is essentially palliative (Grossmann et al., Journal of the National Cancer Institute, 93:1687-97 (2001)). This stage of the disease is referred to as androgen-independent, androgen-refractory, or androgen depletion-independent (ADI) prostate cancer (Roy-Burman et al., Cancer Biol Ther., 4:4-5, (2005)). Although ADI prostate cancer is resistant to androgen ablation, the AR remains critical for the growth and survival of most tumors (Litvinov et al., J Clin Endocrinol Metab., 88:2972-82 (2003)).

### SUMMARY

This document provides methods and materials involved in modulating (e.g., inhibiting or stimulating) the activity of an androgen receptor polypeptide. For example, this document provides methods and materials for identifying molecules that interact with and inhibit or stimulate the activity of an androgen receptor polypeptide. Such molecules can interact with an androgen receptor polypeptide in a manner that requires one or more particular amino acids of the androgen receptor polypeptide. Molecules that modulate androgen receptor activity can be used to treat mammals (e.g., humans) having conditions involving aberrant androgen receptor activity, such as prostate cancer.

In general, one aspect of this document features a method of identifying a molecule capable of inhibiting or activating androgen-independent androgen receptor polypeptide activity. The method comprises, or consists essentially of, determining whether or not a test molecule interacts with the androgen receptor polypeptide in a manner requiring one or more amino acids residues of the androgen receptor polypeptide selected from the group consisting of amino acids residues 181, 182, 435, 438, 439, 101-211, 253-361, and 361-490 of the sequence set forth in SEQ ID NO:1, wherein the presence of the interaction indicates that the test molecule is the molecule.

In another aspect, this document features a method of identifying a molecule capable of inhibiting or activating androgen receptor polypeptide activity. The method comprises, or consists essentially of, (a) contacting cells with a test molecule, wherein the cells comprise: (1) a first nucleic acid comprising a nucleotide sequence that encodes (i) an androgen receptor polypeptide or fragment thereof and (ii) a DNA binding domain, and (2) a second nucleic acid comprising a nucleotide sequence that encodes a reporter gene operably linked to one or more recognition sequences for the DNA binding domain, and (b) determining whether or not the expression level of the reporter gene is decreased or increased compared to the level of expression of the reporter gene in control cells not contacted with the test molecule, wherein a decrease in the expression of the reporter gene indicates that the test molecule is a molecule capable of inhibiting androgen receptor polypeptide activity, and wherein an increase in the expression of the reporter gene indicates that the test molecule is a molecule capable of activating androgen receptor polypeptide activity. The cells can be androgen-independent prostate cancer cells. The test molecule can be a polypeptide. The test molecule can be an antibody. The test molecule can be a small molecule. The first nucleic acid can comprise a nucleotide sequence that encodes a human androgen receptor polypeptide lacking a DNA binding domain. The first nucleic acid can comprise a nucleotide sequence that encodes a Gal4 DNA binding domain.

In another aspect, this document features an isolated nucleic acid molecule comprising, or consisting essentially of, a nucleotide sequence encoding a human androgen receptor polypeptide comprising the amino acid sequence set forth in SEQ ID NO: 1 with the proviso that the amino acid sequence comprises a mutation at an amino acid residue selected from the group consisting of amino acid residues 435, 438, and 439. The amino acid sequence can comprise a substitution at amino acid residue 435. The amino acid sequence can comprise a deletion at amino acid residue 435. The amino acid sequence can comprise an insertion at amino acid residue 435.

In another aspect, this document features an isolated nucleic acid molecule comprising, or consisting essentially of, a nucleotide sequence encoding a human androgen receptor polypeptide comprising the amino acid sequence set forth in SEQ ID NO:1 with the proviso that the amino acid sequence lacks the amino acid sequence at residues 101-211, 253-361, or 361-490.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A, upper panel is a Northern blot analyzing PSA RNA levels in LNCaP or C4-2 cells grown for 48 hours in serum free medium, or medium containing 10% CSS, and stimulated with 1 nM mibolerone (Mib) or vehicle control (EtOH) for an additional 24 hours. One set of cells was re-fed with fresh serum-free medium every six hours to prevent conditioning of the medium. GAPDH RNA expression was analyzed as a control. AR polypeptide expression levels in the LNCaP and C4-2 cells were analyzed concurrently by Western blotting (lower panel). ERK-2 polypeptide expression was analyzed as a control. FIG. 1B, upper panel is a Western blot analyzing AR polypeptide expression in C4-2 cells that were transfected with siRNAs targeted to the AR, or with non-targeted control siRNAs and then maintained in medium containing 5% CSS for an additional 48 hours. ERK-2 polypeptide expression served as a control. PSA RNA levels in the transfected C4-2 cells were analyzed concurrently by Northern blotting (middle panel). As controls, GAPDH RNA levels were analyzed and ribosomal RNA subunits were stained with ethidium bromide. FIG. 1C, upper panel is a schematic diagram of luciferase reporter constructs that were used to transfect LNCaP and C4-2 cells. FIG. 1C, lower panel is a graph plotting luciferase activity in LNCaP and C4-2 cells that were transfected with a luciferase reporter construct depicted in the upper panel, cultured in medium containing 5% CSS for 24 hours following transfection, and then switched to serum-free, phenol red-free medium for an additional 24 hours. Luciferase activity was normalized to the luciferase activity of the -5746 PSA-LUC construct in LNCaP cells.

FIG. 2A is a schematic diagram of luciferase reporter constructs. Bent arrows represent transcription start sites. FIG. 2B is a schematic diagram of the hAR^{Gal4} hybrid polypeptide. FIG. 2C contains graphs plotting luciferase activity in LNCaP cells that were transfected with the PSAenh(ARE)-E4-LUC, PSAenh(GAL4)-E4-LUC, or pG5-LUC reporter construct along with the pM (Gal4 DBD) plasmid or the hAR^{Gal4} construct as indicated, cultured in medium containing 5% CSS for 24 hours following transfection, and then switched to serum-free, phenol red-free medium containing 1 nM mibolerone (Mib) or ethanol (EtOH, vehicle control) for an additional 24 hours. Luciferase activity was normalized to the luciferase activity of the PSAenh(ARE)-E4-LUC construct stimulated with vehicle (EtOH). FIG. 2D contains a series of graphs plotting luciferase activity in C4-2 (top) or LNCaP (bottom) cells that were transfected with the PSAenh(ARE)-LUC, PSAenh(GAL4)-LUC, or pG5-LUC reporter construct along with the pM (Gal4 DBD) plasmid or the hAR^{Gal4} construct as indicated, cultured in medium containing 5% CSS for 24 hours following transfection, and then switched to serum-free, phenol red-free medium for an additional 24 hours. Luciferase activity was normalized to the activity of the PSAenh(GAL4)-LUC construct in the absence of transactivator.

FIG. 3A is a schematic diagram illustrating the location of specific mutations incorporated into the NTD and AF-2 binding surface of the hAR^{Gal4} polypeptide. Brackets indicate substituted residues. FIG. 3B contains graphs plotting fold activation of luciferase activity, relative to the luciferase activity of the PSAenh(GAL4)-LUC construct in the absence of transactivators and androgens, in LNCaP (left panel) and C4-2 (right panel) cells that were transfected with the PSAenh(GAL4)-LUC reporter construct and wild-type, V716R, K720A, or E897K versions of the hAR^{Gal4} construct, cultured in medium containing 5% CSS for 24 hours following transfection, and then switched to serum-free, phenol red-free medium containing 1 nM mibolerone (Mib) or ethanol (EtOH, vehicle control) for an additional 24 hours. FIG. 3C contains graphs plotting fold activation of luciferase activity, relative to the luciferase activity of the PSAenh(GAL4)-LUC construct in the absence of transactivators and androgens, in LNCaP (left panel) and C4-2 (right panel) cells that were transfected with the PSAenh(GAL4)-LUC reporter construct and wild-type, AQNAA (SEQ ID NO:2), AHTAA (SEQ ID NO:3), or AQNAA (SEQ ID NO:2)/AHTAA (SEQ ID NO:3) versions of the hAR^{Gal4} construct as described for FIG. 3B.

FIG. 4A, left panel is a schematic diagram illustrating NTD deletions of hAR^{Gal4}. FIG. 4A, right panels contain graphs plotting fold activation of luciferase activity, relative to the luciferase activity of PSAenh(GAL4)-LUC in the absence of transactivators and androgens, in LNCaP (left graph) and C4-2 (right graph) cells that were transfected with the PSAenh(GAL4)-LUC reporter construct and wild-type, Δ101/211, Δ211/253, Δ253/361, or Δ361/490 versions of the hAR^{Gal4} construct as indicated, cultured in medium containing 5% CSS for 24 hours following transfection, and then switched to serum-free, phenol red-free medium containing 1 nM mibolerone (Mib) or ethanol (EtOH, vehicle control) for an additional 24 hours. FIG. 4B, left panel is a schematic diagram of the wild-type (¹⁵⁸PSTLSLLGPTFPGLSSCSADIKDILSEA¹⁸⁵; SEQ ID NO:4), S159N/S162N (¹⁵⁸PNTLNL¹⁶³; SEQ ID NO:5), I181N/L182N (¹⁷⁸IKDNNSEA¹⁸⁵; SEQ ID NO:6) and Δ101/211 versions of the hAR^{Gal4} polypeptide. FIG. 4B, right panels contain graphs plotting fold activation of luciferase activity, relative to the luciferase activity of the PSAenh(GAL4)-LUC construct in the absence of transactivators and androgens, in LNCaP (left graph) and C4-2 (right graph) cells that were transfected with the PSAenh(GAL4)-LUC reporter construct and wild-type, S159N/S162N, I181N/L182N and Δ101/211 versions of the hAR^{Gal4} construct as described for FIG. 4A. FIG. 4C, left panel is a schematic diagram of the wild-type (³³⁴GTLELPSTLSLYKSGA³⁴⁹; SEQ ID NO:7), S340N/S343N (³³⁹PNTLNL³⁴⁴; SEQ ID NO:5) and A253/361 versions of the hAR^{Gal4} polypeptide. FIG. 4C, right panel contains graphs plotting the fold activation of luciferase activity, relative to the luciferase activity of the PSAenh(GAL4)-LUC construct in the absence of transactivators and androgens, in LNCaP (left graph) and C4-2 (right graph) cells that were transfected with the PSAenh(GAL4)-LUC reporter construct and wild-type, S340N/S343N or Δ253/361 versions of the hAR^{Gal4} construct as described for FIG. 4A.

FIG. 5A, left panel is a schematic diagram of the wild-type (⁴³⁰AASSSWHTLFTAEEG⁴⁴⁴; SEQ ID NO: 8), ⁴³⁵AHTAA⁴³⁹ (SEQ ID NO:3), or Δ361/490 versions of the hAR^{Gal4} polypeptide. FIG. 5A, right panels contain graphs plotting the fold activation of luciferase activity, relative to the luciferase activity of the pG5-LUC construct in the absence of transactivators and androgens, in C4-2 cells that were transfected with the PSAenh(GAL4)-LUC (left) or pG5-LUC (right) reporter construct and the wild-type, ⁴³⁵AHTAA⁴³⁹ (SEQ ID NO:3), or Δ361/490 version of the hAR^{Gal4} construct, cultured in medium containing 5% CSS for 24 hours following transfection, and then switched to serum-free, phenol red-free medium containing 1 nM mibolerone (Mib) or ethanol (EtOH, vehicle control) for an additional 24 hours. FIG. 5B contains a Western blot analyzing hAR^{Gal4} polypeptide expression using an antibody specific for Gal4 in whole cell lysates from mock transfected C4-2 cells, or cells transfected as described in the legend for FIG. 5A. ERK-2 polypeptide expression was analyzed as a control. FIG. 5C, left panel is a schematic diagram illustrating the wild-type hAR^{Gal4} polypeptide (⁴³⁰AASSSWHTLFTAEEG⁴⁴⁴; SEQ ID NO:8) and various mutations in the region of the AR polypeptide encompassing amino acid residues ⁴³⁵WHTLF⁴³⁹ (SEQ ID NO:9). FIG. 5C, right panel is a graph plotting fold activation of luciferase activity, relative to the luciferase activity of the PSAenh(GAL4)-LUC construct in the absence of transactivators and androgens, in C4-2 cells that were transfected with the PSAenh(GAL4)-LUC reporter construct and the wild-type or a ⁴³⁵WHTLF⁴³⁹ (SEQ ID NO:9) mutant version of the hAR^{Gal4} construct as described in the legend for FIG. 5A.

FIG. 6A, top panel is a Northern blot analyzing expression levels of PSA RNA in LNCaP and C4-2 cells that were cultured in medium containing 5% CSS for 24 hours and then shifted to serum free medium containing 1 nM mibolerone (Mib), 10 μM bicalutamide (bic), vehicle control, or combinations of these compounds as indicated for an additional 24 hours. As controls, GAPDH levels were also analyzed on the Northern blot, and ribosomal RNA subunits in the gel were stained with ethidium bromide (middle panel). FIG. 6A, lower panel is a Western blot analyzing AR polypeptide expression levels in LNCaP and C4-2 cells that were treated as described in the legend for FIG. 6A. ERK-2 polypeptide expression levels were analyzed as a control. FIG. 6B contains graphs plotting fold activation of luciferase activity, relative to the luciferase activity of the PSAenh(GAL4)-LUC construct in the absence of transactivators and androgens, in LNCaP and C4-2 cells that were transfected with the PSAenh(GAL4)-LUC reporter construct and the hAR^{Gal4} construct, cultured in medium containing 5% CSS for 24 hours following transfection, and then switched to serum-free, phenol red-free medium containing 1 nM mibolerone (Mib), 10 μM bicalutamide, ethanol (EtOH, vehicle control), or combinations of these compounds as indicated for an additional 24 hours. FIG. 6C is a graph plotting the relative number of viable C4-2 cells, determined using the MTS assay, following culture in medium containing 5% CSS and 10 μM bicalutamide (bic) or ethanol (EtOH, vehicle control) for 24, 48, or 72 hours. FIG. 6D is a schematic diagram depicting AR domains important for ligand-dependent and ligand-independent AR activity. Black boxes with white “+” signs represent positive regulation and white boxes with black “−” signs represent negative regulation. Key amino acid residues within these domains are indicated. The sequence ¹⁷⁸IKDILSEA¹⁸⁵ is set forth in SEQ ID NO:10, and the sequence ⁴³⁵WHTLF⁴³⁹ is set forth in SEQ ID NO:9.

FIG. 7 sets forth the amino acid sequence of the human androgen receptor polypeptide (GenBank accession number P10275; gi|113830; SEQ ID NO:1).

FIG. 8 contains a Northern blot and graphs demonstrating that AR TAU5 plays a differential role in mediating ligand-dependent and ligand-independent AR activity in a cell-based model of prostate cancer progression. Panel A contains a Northern blot analyzing PSA mRNA levels in LNCaP and C4-2 cells that were treated for 24 hours with 1 nM mibolerone (Mib) or vehicle control (EtOH) in serum-free medium. Glyceraldehyde-3-phosphate dehydrogenase (GAPDH) mRNA levels and ethidium bromide staining of ribosomal RNA subunits are shown as controls. AR polypeptide expression was concurrently determined by Western blot. ERK-2 polypeptide expression is shown as a control. Panel B, left panel contains a schematic illustration of a Gal4-based AR tethering system to allow AR functional studies in AR-expressing cells. Modularity of the AR polypeptide is indicated, as well as the location of TAU5. The hinge (h) region between the DBD and LBD is indicated. Other domains are described in the text. Panel B, right panel contains graphs plotting luciferase activity in LNCaP and C4-2 cells that were transfected with ARE-driven or GAL4-driven PSA-based reporter constructs (depicted in the left panel) along with wild-type (WT) AR^{Gal4} or AR^{Gal4} ΔTAU5, as indicated. Cells were grown in serum-free medium and treated with 1 nM Mib or EtOH for 24 hours. Data represent the mean±S.E. from at least three independent experiments, each performed in duplicate. Values are shown relative to the activity of the GAL4-based reporter construct in the absence of androgens and transactivator, which was arbitrarily set to 1. Panel C contains graphs plotting luciferase activity in LNCaP and C4-2 cells that were transfected with MMTV-Luc, non-targeted control (ctrl), or AR-targeted siRNAs, and wild-type or TAU5-deleted (ΔTAU5) versions of AR harboring silent mutations in the siRNA target site (denoted AR^{sr}) as indicated. Luciferase activity was determined as described for panel B. Lysates were analyzed by Western blot for AR polypeptide expression. ERK-2 polypeptide expression is shown as a control. The ligand-dependent (LNCaP) and ligand-independent (C4-2) activity of endogenous AR on the MMTV promoter was arbitrarily set to 100%. Panel D contains a graph plotting luciferase activity in 22Rv1 cells that were transfected as described for panel C. Luciferase activity, AR polypeptide expression, and ERK-2 polypeptide expression were determined as described for panel C. Values are shown relative to the ligand-independent activity of hAR^{sr}, which was arbitrarily set to 100%.

FIG. 9 contains graphs and Western blots demonstrating that TAU5 is required for ligand-hypersensitive AR activity in ADI prostate cancer cells. Panel A contains graphs plotting luciferase activity in LNCaP and C4-2 cells that were transfected with wild-type (WT) and ΔTAU5 versions of AR^{Gal4} in conjunction with PSAenh(GAL4)-LUC. The cells were treated under serum-free conditions with 2 pM-0.2 nM dihydrotestosterone (DHT) for 24 hours, and luciferase activity was determined. The data represent the mean±S.E. from at least three independent experiments, each performed in duplicate. The activity of wild-type hAR^{Gal4} in response to 0.2 nM DHT was arbitrarily set to 100%. Panel B contains graphs plotting luciferase activity in LNCaP and C4-2 cells in which endogenous AR expression was knocked-down via AR-targeted siRNA. The cells were co-transfected with constructs encoding WT and ΔTAU5 versions of siRNA-resistant (AR^{sr}) along with MMTV-LUC as indicated. The cells were treated and the luciferase activity was determined as described for panel A. Panel C contains graphs plotting luciferase activity in LNCaP and C4-2 cells that were transfected with PSAenh(GAL4)-LUC along with wild-type, CTD-deleted (NTD^{Gal4}), or CTD/TAU5-deleted (NTD^{Gal4} TAU5) versions of AR^{Gal4} as indicated and treated with 1 nM mibolerone (Mib) or vehicle control (ethanol, EtOH) for 24 hours. Luciferase activity was determined as described for panel A. The values are shown relative to the activity of the GAL4-based reporter construct in the absence of androgens and transactivator, which was arbitrarily set to 1. Lysates were analyzed by Western blot for AR polypeptide expression. The discrete polypeptide species recognized by the NTD-targeted AR antibody are indicated.

FIG. 10 contains schematic illustrations, graphs, and Western blots demonstrating that a ⁴³⁵WHTLF⁴³⁹ (SEQ ID NO:9) motif within the TAU5 domain is selectively required for ligand-independent AR activity in ADI prostate cancer cells. Panel A contains an amino acid sequence of AR TAU5 (SEQ ID NO: 11). Propensity for α-helical (h) or β-sheet (e) secondary structure was determined using the nnpredict algorithm. Panel B contains a graph plotting luciferase activity in C4-2 cells that were transfected with wild-type (WT), Δ393/420, and Δ420/449 versions of AR^{Gal4} in conjunction with PSAenh(GAL4)-LUC. Cells were maintained for 48 hours in the absence of androgens, and luciferase activity was determined. Data represent the mean±S.E. from at least three independent experiments, each performed in duplicate. Values are shown relative to the activity of the GAL4-based reporter construct in the absence of androgens and transactivator, which was arbitrarily set to 1. Panel C contains a graph plotting luciferase activity in C4-2 cells that were transfected with PSAenh(GAL4)-LUC and WT AR^{Gal4} or versions of AR^{Gal4} harboring single, double, or triple alanine substitutions at W435, L438, and F439, as indicated. The relative locations of W425, L438, and F439 are depicted in the context of a helical wheel. Luciferase activity was determined as described for panel B. Values are shown relative to the activity of wild-type (WT) hAR^{Gal4} in the absence of androgens and transactivator, which was arbitrarily set to 100%. (*P≦0.01; **P≦0.0001). The sequence ⁴³⁰AASSSWHTLFTAEEG⁴⁴⁴ is set forth in SEQ ID NO:8. Panel D contains graphs plotting luciferase activity in LNCaP and C4-2 cells that were transfected with MMTV-Luc and non-targeted control (ctrl) or AR-targeted siRNAs as indicated. Wild-type or ⁴³⁵AHTAA⁴³⁹ (SEQ ID NO:3) versions of AR were expressed from vectors harboring silent mutations in the siRNA target site (denoted AR^{sr}) as indicated. Cells were treated under serum-free conditions with 1 nM Mib or EtOH for 24 hours. Luciferase activity was determined as described for panel B. The ligand-dependent (LNCaP) and ligand-independent (C4-2) activity of hAR^{sr} was arbitrarily set to 100%. Lysates were analyzed by Western blot for AR polypeptide expression. ERK-2 polypeptide expression is shown as a control.

FIG. 11 contains graphs demonstrating that the AR ⁴³⁵WHTLF⁴³⁹ (SEQ ID NO:9) motif mediates ligand-independent TAU5 transcriptional activity. Panel A contains a graph plotting luciferase activity in C4-2 cells that were transfected with wild-type (WT), ΔTAU5, and AHTAA (SEQ ID NO:3) mutant versions of hAR^{Gal4}, as well as ΔTAU5 versions of hAR^{Gal4} with 21 amino acid inserts encompassing wild-type (SAAASSSWHTLFTAEEGQLYG; SEQ ID NO:12) or AHTAA (SEQ ID NO:3) mutant (SAAASSSAHTAATAEEGQLYG; SEQ ID NO:13) versions of the ⁴³⁵WHTLF⁴³⁹ (SEQ ID NO:9) domain as indicated. PSAenh(GAL4)-LUC was employed as a reporter for these experiments. Cells were maintained for 48 hours in the absence of androgens, and luciferase activity was determined. The data represent the mean±S.E. from at least three independent experiments, each performed in duplicate. Values are shown relative to the activity of the GAL4-based reporter construct in the absence of androgens and transactivator, which was arbitrarily set to 1. Lysates were analyzed by Western blot for hAR^{Gal4} polypeptide expression with Gal4-specific antibodies. Panel B contains a graph plotting luciferase activity in C4-2 cells that were transfected with WT or ΔTAU5 versions of hARGal4, or ΔTAU5 versions of hAR^{Gal4} harboring 1, 2, or 3 copies of a 21 amino acid insert encompassing the AR WHTLF (SEQ ID NO:9) motif. PSAenh(GAL4)-LUC was employed as a reporter for these experiments. Assays were performed as described for panel A. Panel C contains a graph plotting luciferase activity in LNCaP cells that were transfected with hAR^{Gal4}-based constructs as described for panels A and B. Cells were treated under serum-free conditions with 1 nM Mib or EtOH for 24 hours. (*P≦0.05).

FIG. 12 contains graphs, schematic illustrations, and a Western blot demonstrating that AR ⁴³⁵WHTLF⁴³⁹ (SEQ ID NO:9) represents a novel ligand-independent transactivation motif Panel A contains a graph plotting luciferase activity in C4-2 cells that were transfected with wild-type (WT) or AF-2 mutant (V716R, K720A, E897K) versions of hAR^{Gal4}, ΔTAU5 hAR^{Gal4}, and ΔTAU5 hAR^{Gal4} with a 21 amino acid insert (SEQ ID NO: 12) encompassing the AR WHTLF (SEQ ID NO:9) motif and a wild-type or mutant AF-2 (V716R, K720A, E897K) domain as indicated. PSAenh(GAL4)-LUC was employed as a reporter for these experiments. Cells were maintained for 48 hours in the absence of androgens, and luciferase activity was determined. The data represent the mean±S.E. from at least three independent experiments, each performed in duplicate. Values are shown relative to the activity of the GAL4-based reporter construct in the absence of androgens and transactivator, which was arbitrarily set to 1. Lysates were analyzed by Western blot for hAR^{Gal4} polypeptide expression with Gal4-specific antibodies. Panel B contains graphs plotting luciferase activity in LNCaP and C4-2 cells that were transfected with constructs encoding fusion polypeptides containing the Gal4 DBD and 1, 2, or 3 copies of a 21 amino acid insert encompassing wild-type (SEQ ID NO: 14) or AHTAA (SEQ ID NO:3) mutant (SEQ ID NO: 15) versions of the ⁴³⁵WHTLF⁴³⁹ (SEQ ID NO:9) domain as indicated. pG5-LUC was employed as a reporter for these experiments. Cells were transfected and luciferase activity determined as described for panel A.

FIG. 13 is a graph plotting luciferase activity in LNCaP cells that were transfected with ARE-driven or GAL4-driven PSA-based reporter constructs along with wild-type (WT) AR^{Gal4} or AR^{Gal4} ΔTAU5 as indicated. Cells were grown in serum-free medium for 48 hours, and luciferase activity was determined. Data represent the mean±S.E. from at least three independent experiments, each performed in duplicate.

FIG. 14 is a graph plotting luciferase activity in C4-2 cells that were transfected with MMTV-LUC along with wild-type and ΔTAU5 versions of AR^{sr} as indicated, and were treated with 1 nM Mib or vehicle control (EtOH) for 24 hours. Luciferase activity was determined. Data represent the mean±S.E. from at least three independent experiments, each performed in duplicate.

FIG. 15, panel A contains a Northern blot analyzing PSA mRNA levels in ADI 22Rv1 cells that were grown in serum-free conditions for 48 hours and treated with 1 nM mibolerone (Mib), 10 μM casodex (CDX), or combinations of these compounds for an additional 24 hours. Glyceraldehyde-3-phosphate dehydrogenase (GAPDH) mRNA levels and ethidium bromide staining of ribosomal RNA subunits are shown as controls. AR polypeptide expression was concurrently determined by Western blot. ERK-2 polypeptide expression is shown as a control. Panel B contains a Northern blot analyzing PSA mRNA levels in 22Rv1 cells that were transfected in androgen-free conditions with separate siRNAs targeted to the AR or a non-targeted control (ctrl) siRNA. Levels of GAPDH mRNAs, ribosomal RNA subunits, as well as AR and ERK-2 polypeptides also were determined as described for panel A. Casodex selectively inhibits AF-2-dependent but not AF-2-independent AR activity (Dehm and Tindall, J Biol Chem, 281:27882-27893 (2006)). Ligand-independent PSA expression in 22Rv1 cells is AR dependent (Panel B), but resistant to casodex (Panel A), indicating that this activity is AF-2 independent.

### DETAILED DESCRIPTION

This document provides methods and materials involved in modulating (e.g., inhibiting or stimulating) the activity of an androgen receptor polypeptide. As described herein, this document provides methods and materials for identifying molecules that interact with and modulate the activity of an androgen receptor polypeptide. Molecules can interact with and modulate the activity of an androgen receptor polypeptide in a manner that requires one or more particular amino acids of the androgen receptor polypeptide.

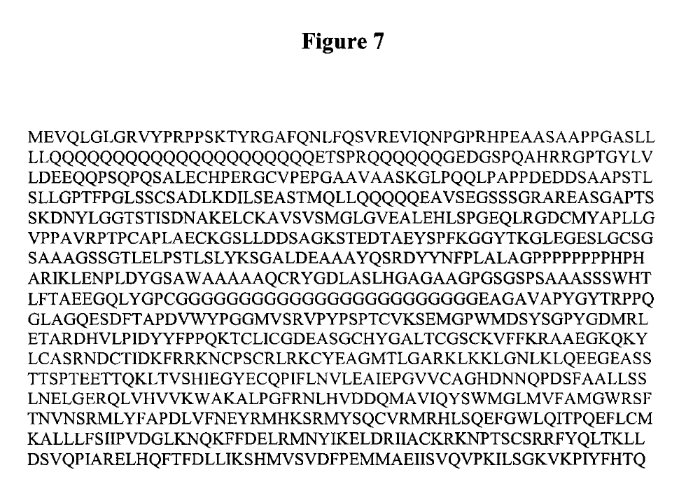

The activity of any androgen receptor polypeptide can be modulated including, without limitation, a native or mutant androgen receptor polypeptide. Examples of androgen receptor polypeptides include, without limitation, human androgen receptor polypeptides (e.g., GenBank® accession number P10275 (gi|13830); FIG. 7; SEQ ID NO: 1), canine androgen receptor polypeptides (e.g., GenBank® accession number NP_{—}001003053.1 (gi|50978706)), chimpanzee androgen receptor polypeptides (e.g., GenBank® accession numbers NP_{—}001009012 (gi|57113915) and XP_{—}521101.1 (gi|55663272)), rat androgen receptor polypeptides (e.g., GenBank® accession number NP_{—}036634.1 (gi|6978535)), and mouse androgen receptor polypeptides (e.g., GenBank® accession number NP_{—}038504.1 (gi|7304901)).

The activity of an androgen receptor polypeptide can be modulated in any cell having an androgen receptor polypeptide. For example, the activity of an androgen receptor polypeptide can be inhibited in androgen-dependent and androgen-independent prostate cancer cells. In addition, the activity can be modulated in the presence or absence of androgens.

Any type of molecule that interacts with an androgen receptor polypeptide can be used to modulate the activity of the androgen receptor polypeptide. For example, a small molecule, a peptidomimetic, a polymer, or an antibody can be used to modulate the activity of an androgen receptor polypeptide. The interaction between the molecule and the androgen receptor polypeptide can be any type of interaction, e.g., a covalent interaction, a non-covalent interaction, a charge-charge interaction, a hydrogen-bonding interaction, or any combination of interactions.

Any method can be used to identify a molecule that interacts with an androgen receptor polypeptide. For example, a reporter assay system can be used to identify a molecule that interacts with an androgen receptor polypeptide. Such an assay can be based on a recombinant nucleic acid construct comprising a nucleic acid encoding an androgen receptor polypeptide comprising a DNA binding domain, and a second recombinant nucleic acid construct comprising a reporter gene operably linked to one or more recognition sequences for the DNA binding domain in the androgen receptor polypeptide. A nucleic acid encoding an androgen receptor polypeptide comprising a DNA binding domain and a reporter gene operably linked to one or more recognition sequences for the DNA binding domain can also be included in a single recombinant nucleic acid construct. As used herein, the term “operably linked” refers to positioning of a regulatory region and a sequence to be transcribed in a nucleic acid so as to influence transcription of the sequence. Cells can be transiently or stably transfected with the recombinant nucleic acid construct(s) comprising the androgen receptor polypeptide and the reporter gene, and the cells can be mock-treated or treated with a test molecule. The activity of the reporter gene can be analyzed and compared in the mock-treated and molecule-treated cells. An increased or reduced level of reporter gene activity in the molecule-treated cells as compared to the mock-treated cells can indicate that the molecule interacts with the androgen receptor polypeptide. Any DNA binding domain and corresponding recognition sequence can be used. For example, a Gal4 DNA binding domain can be used with Gal4 recognition sequences. Any reporter gene can be used including, without limitation, luciferase, green fluorescent protein, and β-gal. Any cells can be used, regardless of whether they contain endogenous androgen receptor polypeptides. For example, androgen-dependent or androgen-independent prostate cancer cells can be used. In some cases, androgen-dependent cells can be used and the cells can be treated with androgen in addition to the molecule being tested. A reporter assay system can be used to screen large number of molecules, such as small molecule libraries, for molecules that can interact with an androgen receptor polypeptide. The dose response and time course of alterations in androgen receptor activity can also be investigated.

In some cases, a Biacore (Uppsala, Sweden) system based on surface plasmon resonance can be used to identify a molecule that can interact with an androgen receptor polypeptide. An androgen receptor polypeptide or any fragment of an androgen receptor polypeptide (e.g., a fragment consisting of amino acids corresponding to amino acids 101-211, 253-361, or 361-490 of the amino acid sequence set forth in FIG. 7 and SEQ ID NO: 1) can be immobilized on a sensor surface, and any molecular interaction with the immobilized polypeptide can be monitored using the surface plasmon resonance phenomenon. For example, interactions with small molecules, peptides, or multiple sub-unit polypeptide complexes can be monitored. The specificity, affinity, and kinetics of an interaction can be determined.

In some cases, a yeast two-hybrid technique known to those of skill in the art can be used to identify polypeptides that can interact with an androgen receptor polypeptide or a fragment of an androgen receptor polypeptide. In some cases, an androgen receptor polypeptide or fragment thereof can be screened against a phage display polypeptide library and phage displaying a polypeptide that can interact with the androgen receptor polypeptide or androgen receptor polypeptide fragment can be amplified and sequenced (see, e.g., Nowakowski et al., Stem Cells, 22(6):1030-8 (2004)) to identify the polypeptide. Phage display polypeptide library kits are commercially available, e.g., from New England Biolabs (Ipswich, Mass.).

In some cases, an androgen receptor polypeptide or a particular fragment of an androgen receptor polypeptide can be used to raise an antibody against the androgen receptor polypeptide or particular fragment thereof. An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type (e.g., IgG, IgM, IgD, IgA or IgY), class (e.g., IgG 1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-androgen receptor antibody can bind to androgen receptor polypeptides at an affinity of at least 10⁴ mol⁻¹, e.g., at least 10⁵, 10⁶, 10⁷, 10⁸, 10⁹, 10¹⁰, 10¹¹, or 10¹² mol⁻¹. In addition, an anti-androgen antibody can be conjugated to a membrane-translocating sequence normally found in signaling polypeptides such as growth factors to allow the antibody to translocate across cell-surface membranes into the cytoplasm and bind to intracellular targets (see, e.g., the website of InNexus Biotechnology, Inc., Scottsdale, Ariz., on the World Wide Web at innexusbiotech.com).

In some cases, a computational method can be used in the identification of a molecule that interacts with an androgen receptor polypeptide. In particular, a computational method can be used to model interactions between molecules and particular amino acids or amino acid regions of an androgen receptor polypeptide. In some cases, a computational method can be used to model interactions between an androgen receptor polypeptide and small molecules in a physical or virtual library. Interactions between an androgen receptor polypeptide and candidate molecules identified computationally can be tested using any method, including methods described herein.

A molecule that interacts with an androgen receptor polypeptide can be used to identify additional molecules that interact with the androgen receptor polypeptide. For example, a molecule that interacts with an androgen receptor polypeptide can be labeled and used in a binding assay to identify additional molecules that interact with the androgen receptor polypeptide. In some cases, a polypeptide that interacts with an androgen receptor polypeptide can be radioactively or fluorescently labeled and used in a competitive binding assay to identify small molecules that interact with the androgen receptor polypeptide.

Once a molecule that interacts with an androgen receptor polypeptide is identified, it can be determined whether or not the interaction between the molecule and the androgen receptor polypeptide requires a particular region or amino acid(s) of the androgen receptor polypeptide. For example, a nonconservative substitution of one or more amino acids of the androgen receptor polypeptide can be generated, or one or more regions of the androgen receptor polypeptide can be deleted, as described herein, and the ability of the molecule to interact with the mutant androgen receptor polypeptide can be tested. A nonconservative substitution or deletion of an amino acid residue of an androgen receptor polypeptide that does not interfere with the interaction between a molecule and the androgen receptor polypeptide indicates that the amino acid residue is not required for the interaction. Conversely, a mutation in an amino acid residue that interferes with the interaction between a molecule and the androgen receptor polypeptide indicates that the mutated amino acid is required for the interaction.

A molecule that interacts with an androgen receptor polypeptide can be tested to determine whether the molecule modulates the activity of an androgen receptor polypeptide. Any method can be used to determine whether the molecule modulates the activity of an androgen receptor polypeptide. For example, a reporter assay system described above can be used to determine whether the molecule modulates the activity of an androgen receptor polypeptide. In some cases, a molecule that interacts with an androgen receptor polypeptide can be tested in an animal model of prostate cancer or in androgen-independent prostate cancer cells to determine whether the molecule modulates the activity of an androgen receptor polypeptide. The dose response and time course of the effect can also be determined.

The activity of an androgen receptor polypeptide can be modulated (e.g., inhibited or stimulated) using a molecule that interacts with the androgen receptor polypeptide, where the interaction requires one or more amino acids of the androgen receptor polypeptide. For example, the activity of an androgen receptor polypeptide can be inhibited using a molecule that interacts with the androgen receptor polypeptide, where the interaction requires one or more amino acids of the androgen receptor polypeptide corresponding to amino acids 181, 182, 435, 438, 439, 716, 720, 897, 101-211, 253-361, or 361-490 of the amino acid sequence of the human androgen receptor polypeptide set forth in FIG. 7 and SEQ ID NO: 1. In some cases, the activity of an androgen receptor polypeptide can be stimulated using a molecule that interacts with the androgen receptor polypeptide, where the interaction requires one or more amino acids of the androgen receptor polypeptide corresponding to amino acids 435, 438, 439, 211-253, or 361-490 of the amino acid sequence of the human androgen receptor polypeptide set forth in FIG. 7 and SEQ ID NO: 1. In some cases, the activity of the androgen receptor polypeptide can be androgen-dependent or androgen-independent.

Modulation of androgen receptor activity can result in an increase in androgen receptor activity or a partial or complete reduction in androgen receptor activity. For example, inhibition of androgen receptor activity can result in a reduced or undetectable level of expression of a gene, such as PSA, that is regulated by androgen receptor polypeptides. In some cases, stimulation of androgen receptor activity can result in an increased level of expression of a gene, such as PSA, that is regulated by androgen receptor polypeptides. In some cases, inhibition of androgen receptor activity can result in disruption of a polypeptide-polypeptide interaction between two androgen receptor polypeptides or between an androgen receptor polypeptide and another polypeptide, such as a CTNNB1, CAV1, FHL2, ARA70, SRC-1, TIF2, or KIAA1377 polypeptide.

Molecules that modulate androgen receptor activity can be used to treat mammals (e.g., humans, dogs, chimpanzees, rats, mice, and chickens) having conditions involving a defect in an androgen receptor including, without limitation, androgen-insensitivity syndrome, Reifensteins syndrome, spinal and bulbar muscular atrophy (Kennedy's disease), testicular feminization, spinobulbar muscular atrophy, and virilism. For example, a molecule that inhibits the activity of an androgen receptor polypeptide can be used to treat mammals (e.g., humans) having prostate cancer. In some cases, a molecule that inhibits the activity of an androgen receptor polypeptide in androgen-independent prostate cancer cells can be used to treat mammals (e.g., humans) having androgen-independent prostate cancer.

A molecule that modulates the activity of an androgen receptor polypeptide can be formulated for administration by any route. For example, a molecule that modulates androgen receptor activity can be formulated for oral administration or administration by injection (e.g., subcutaneous, intravenous, or intramuscular injection, or injection directly into a tumor), and can be administered once or more than once, e.g., weekly, monthly, three times a year, or yearly. Administration can be intermittent or continuous over any course of time, such as the remainder of a mammal's lifetime. The dose can be any dose effective to modulate androgen receptor activity without producing significant toxic side effects.

Molecules that modulate the activity of an androgen receptor polypeptide can be administered in the presence or absence of agents that stabilize biological activity. For example, a molecule that modulates the activity of an androgen receptor polypeptide can be pegylated, acetylated, or both. In some cases, a molecule that modulates the activity of an androgen receptor polypeptide can be covalently attached to oligomers, such as short, amphiphilic oligomers that enable oral administration or improve the pharmacokinetic or pharmacodynamic profile of the conjugated polypeptide. The oligomers can include water soluble polyethylene glycol (PEG) and lipid soluble alkyls (short chain fatty acid polymers). See, for example, International Patent Application Publication No. WO 2004/047871.

This document also provides isolated nucleic acids encoding a human androgen receptor polypeptide comprising the amino acid sequence set forth in SEQ ID NO: 1 with the proviso that the amino acid sequence comprises a mutation (e.g., a mutation described herein) at one or more amino acid residues. An example of a nucleic acid encoding a human androgen receptor polypeptide comprising the amino acid sequence set forth in SEQ ID NO:1 is set forth in GenBank® under GI number 178627. The term “mutation” as used herein with respect to polypeptides includes insertions of one or more amino acids, deletions of one or more amino acids, amino acid substitutions, and combinations thereof. For example, this document provides isolated nucleic acids encoding a human androgen receptor polypeptide comprising the amino acid sequence set forth in SEQ ID NO: 1 with the proviso that said amino acid sequence comprises a mutation at an amino acid residue selected from the group consisting of amino acid residues 435, 438, and 439. In some cases, an isolated nucleic acid provided herein can encode a human androgen receptor polypeptide comprising the amino acid sequence set forth in SEQ ID NO: 1 with the proviso that the amino acid sequence comprises a substitution at amino acid residue 435, a deletion at amino acid residue 435, or an insertion at amino acid residue 435. In some cases, an isolated nucleic acid molecule provided herein can comprise a nucleotide sequence encoding a human androgen receptor polypeptide comprising the amino acid sequence set forth in SEQ ID NO: 1 with the proviso that the amino acid sequence lacks the amino acid sequence at residues 101-211, 253-361, or 361-490.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

Typically, isolated nucleic acids provided herein are at least about 20 nucleotides in length. For example, a nucleic acid can be about 20-30 (e.g., 20, 21, 22, 23, 24, 25, 26, 27, 28, 29, or 30 nucleotides in length), 20-50, 50-100, or greater than 100 nucleotides in length (e.g., greater than 150, 200, 250, 300, 350, 400, 450, 500, 750, or 1000 nucleotides in length). Isolated nucleic acids provided herein can be in a sense or antisense orientation, can be single-stranded or double-stranded, can be complementary to a nucleic acid encoding an androgen receptor polypeptide (e.g., SEQ ID NO: 1), and can be DNA, RNA, or nucleic acid analogs. Nucleic acid analogs can be modified at the base moiety, sugar moiety, or phosphate backbone to improve, for example, stability, hybridization, or solubility of the nucleic acid.

Isolated nucleic acids can be produced by standard techniques, including, without limitation, common molecular cloning and chemical nucleic acid synthesis techniques.

Isolated nucleic acids provided herein can be used, e.g., to determine whether or not the interaction between a molecule and an androgen receptor polypeptide requires a particular region or amino acid(s) of the androgen receptor polypeptide. For example, an isolated nucleic acid encoding an androgen receptor polypeptide having one or more regions deleted relative to a wild-type androgen receptor polypeptide, or comprising a nonconservative substitution of one or more amino acids of the androgen receptor polypeptide, can be generated. The isolated nucleic acid can be incorporated into a vector (e.g., a vector that is a component of a reporter assay system described herein), and transfected into cells such that the mutant androgen receptor polypeptide is expressed in the cells. Cells expressing a mutant androgen receptor polypeptide can be used to determine whether or not the interaction between a molecule and an androgen receptor polypeptide requires a region or amino acid(s) that differs between the wild-type and the mutant androgen receptor polypeptide. For example, cells comprising a nucleic acid encoding a mutant androgen receptor polypeptide comprising a DNA binding domain and a reporter gene operably linked to one or more recognition sequences for the DNA binding domain as described herein can be used to determine whether or not the interaction between a molecule and an androgen receptor polypeptide requires a region or amino acid(s) that differs between the wild-type and the mutant androgen receptor polypeptide. A nonconservative substitution or deletion of an amino acid residue of an androgen receptor polypeptide that does not interfere with the interaction between a molecule and the androgen receptor polypeptide can indicate that the amino acid residue is not required for the interaction. Conversely, a mutation in an amino acid residue that interferes with the interaction between a molecule and the androgen receptor polypeptide can indicate that the mutated amino acid is required for the interaction.

The invention will be further described in the following examples, which do not limit the scope of the invention described in the claims.

### EXAMPLES

### Example 1

### Materials and Methods

Cell Lines and Culture Conditions

Androgen dependent LNCaP cells were purchased from the American Type Culture Collection (ATCC, Manassas, Va.). The androgen depletion independent (ADI) LNCaP-derived C4-2 cell line was purchased from UroCor (Oklahoma City, Okla.). The cells were grown in RPMI 1640 medium supplemented with 10% fetal bovine serum (FBS), 100 U/mL penicillin, 100 μg/mL streptomycin, and 0.25 μg/mL amphotericin B. The cells were maintained at 37° C. and 5% CO₂. LNCaP cells between passage 29 and 45, and C4-2 cells between passage 34 and 50 were used for experiments.

For androgen response experiments, LNCaP and C4-2 cells were grown in serum-free medium or medium supplemented with 10% charcoal stripped (steroid depleted) serum (CSS) for 48 hours. The growth medium was replaced with serum-free medium, or medium with 10% CSS, containing 1 nM mibolerone (BIOMOL, Plymouth Meeting, Pa.), 10 μM bicalutamide (AstraZeneca, Wilmington, Del.), ethanol, or combinations of these compounds. The cells were cultured for an additional 24 hours and then harvested. For C4-2 siRNA-transfection experiments, culture medium was replaced 24 hours post-transfection with RPMI containing 5% CSS. The cells were harvested after 48 hours.

Plasmid Constructs

The SV40-Renilla luciferase reporter vector and the pG5-LUC vector were purchased from Promega (Madison, Wis.). All PSA promoter-based fragments contained the firefly luciferase reporter pGL3-Basic backbone (Promega). The full-length PSA promoter luciferase reporter construct, −5746 PSA-LUC, contained a 5.8 kb genomic fragment from the PSA locus, and was obtained from Dr. Charles Young, Mayo Clinic, Rochester, Minn. The −5746 PSA-LUCΔEco/Pst construct was generated by digestion of the −5746 PSA-LUC construct with EcoRI/PstI, creation of blunt ends, and re-ligation. The sPSA-luc and pN/H constructs, referred to herein as PSAenh-LUC and PSAcore-LUC, respectively, are described elsewhere (Yeung et al., J Biol Chem., 275:40846-55 (2000)), and were obtained from Dr. Lelund Chung, Emory University, GA. The PSAenh(ARE)-E4-LUC and S-All constructs also are described elsewhere (Huang et al., J Biol Chem., 274:25756-68 (1999)), and were obtained from Dr. Michael Carey, University of California, Los Angeles. The S-All construct is referred to herein as PSAenh(GAL4)-E4-LUC. The PSAenh(GAL4)-LUC is described by Debes et al. (Cancer Res., 65:5965-73 (2005)). The Gal4 expression plasmid, pM, was purchased from Clontech (Mountain View, Calif.).

Generation of Constructs

The p5HBhAR-A construct contained the full-length human androgen receptor (hAR) cDNA cloned into the pCMV5 expression vector, and was obtained from Dr. Frank French, University of North Carolina at Chapel Hill. The hAR^{Gal4} construct was generated by replacing the hAR DNA binding domain (DBD) with the Gal4 DBD. A MluI site was generated at the 5′ end of the AR DBD in the p5HBhAR-A construct using a QuikChange site-directed mutagenesis kit (Stratagene, La Jolla, Calif.) and the mutagenic primers ARDBD5′mutFWD (5′-CCACCCCAGAAGACGCGTCTGATCTGTGG AGATGAAGC-3′; SEQ ID NO:16) and ARDBD5′mutREV (5′-GCTTCATCTC CACAGATCAGACGCGTCTTCTGGGGTGG-3′; SEQ ID NO: 17). A SacII site was created at the 3′ end of the AR DBD via mutagenesis using the mutagenic primers ARDBD3′mutFWD (5′-GGGATGACTCTGGGACCGCGGAAGCTGA AGAAACTTGG-3′; SEQ ID NO:18) and ARDBD3′mutREV (5′-CCAAGTTTCTT CAGCTTCCGCGGTCCCAGAGTCATCCC-3′; SEQ ID NO:19). The Gal4 DBD was amplified from the pM plasmid using the primers GAL4DBDMluIFWD (5′-GAAAGA CGCGTCTACTGTCTTC-3′; SEQ ID NO:20) and GAL4DBDSacIIREV (5′-AATTCC CGCGGTACAGTCAAC-3′; SEQ ID NO:21). Following amplification, the Gal4 DBD was digested with MluI/SacII and cloned into the MluI/SacII sites flanking the AR DBD in the p5HBhAR-A construct. This version of the AR contained 24 CAG repeats in the AR NTD; however, amino acid residues were numbered based on an AR containing 22 CAG repeats and a wild-type DBD to maintain consistency with the literature. To generate AR^{Gal4} N-terminal domain (NTD) deletions, successive QuikChange site directed mutagenesis reactions were performed to create in-frame BssHII sites at amino acid positions 101 and 211, 211 and 253, 253 and 361, or 361 and 490. The resulting ARGal4 BssHII double mutants were digested with BssHII and re-ligated to generate the AR^{Gal4} deletions Δ101/211, Δ211/253, Δ253/361, and Δ361/490. QuikChange site directed mutagenesis was used to create the ²³AQNAA²⁷ (SEQ ID NO:2), ¹⁵⁸PNTLNL¹⁶³ (SEQ ID NO:5), ¹⁷⁸IKDNNSEA¹⁸⁵ (SEQ ID NO:6), ³³⁹PNTLNL³⁴⁴ (SEQ ID NO:5), ⁴³⁵AHTAA⁴³⁹ (SEQ ID NO:3), ²³AQNAA²⁷ (SEQ ID NO:2)/⁴³⁵AHTAA⁴³⁹ (SEQ ID NO:3), V716R, K720A, and E897K mutant versions of the AR^{Gal4} construct. All deletion and point-mutant constructs were verified by sequencing and tested for expression by transfection and Western blotting with anti-Gal4 antibodies.

Transient Transfections

In experiments directly comparing the activities of reporter constructs in LNCaP and C4-2 cells, the cells were transfected with 1.5 μg of a promoter-LUC construct and 0.5 μg of the SV40-Renilla vector using 10 μL of Superfect reagent (Qiagen, Valencia, Calif.). Cells were seeded the previous day at a density of 5×10⁵ cells per well in 6-well plates. Transfections were allowed to proceed for two hours followed by addition of two mL of fresh medium containing 5% CSS. The same protocol was used to transfect cells with the PSAenh(ARE)-E4-LUC or PSAenh(GAL4)-E4-LUC reporter construct, except that cells were transfected using 1 μg of a promoter-LUC reporter construct, 0.5 μg of the SV40-Renilla vector, and 0.2 or 0.5 μg of either the pM plasmid or the AR^{Gal4} construct and pbluescript vector to a total DNA content of 2 μg In experiments with the PSAenh(ARE)-LUC, PSAenh(GAL4)-LUC, or pG5-LUC reporter construct, cells were transfected using 0.875 μg of a promoter-LUC reporter construct, 0.25 μg of the SV40-Renilla vector, and 13.7 or 54.7 ng of either the pM plasmid or the hAR^{Gal4} construct and pbluescript vector to a total DNA content of 2 μg. LNCAP cells were also transfected with the pG5-LUC and PSAenh(ARE)-LUC constructs using electroporation. In a typical electroporation experiment, 3×10⁶ cells were suspended in 350 μL of medium containing 5% CSS and combined with 50 μL of a DNA mixture containing 5.25 μg of a promoter-LUC reporter, 1.5 μg of the SV40-Renilla vector, 328 or 82 ng of either the pM plasmid or the hAR^{Gal4} construct and pbluescript vector to a total DNA content of 12 μg. The mixture of cells and DNA was subjected to a 305 V electrical pulse for 10 milliseconds in cuvettes with a 4 mm gap-width (BTX) using a BTX ElectroSquare Electroporator. The cells were then plated in medium containing 5% CSS. The medium was aspirated 24 hours post-transfection and replaced with serum-free, phenol red-free medium containing 1 nM mibolerone, 10 μM bicalutamide, ethanol (vehicle control), or combinations of these compounds. The cells were harvested after an additional 24 hours. The transfection efficiency was typically 50-70%. Firefly and Renilla luciferase reporter activities were detected using a Dual Luciferase Assay (Promega). The activities were normalized by dividing the firefly luciferase activity by the Renilla luciferase activity of samples cultured in the absence of androgens. The data are presented as the mean±S.E.M. from at least three independent experiments, each performed in duplicate.

For siRNA transfections, C4-2 cells were seeded at a density of 1.8×10⁶ cells on 10 cm dishes in antibiotic-free RPMI 1640 containing 10% FBS. Cells were transfected 24 hours later with 500 pmol of a pool of four siRNAs targeted to the AR (AR SmartPool; Dharmacon, Lafayette, Colo.), or a pool of four non-targeted, control siRNAs (Dharmacon) using 25 μg Lipofectamine (Invitrogen, Carlsbad, Calif.) according to the manufacturer's protocol.

RNA Extraction and Northern Blot Analysis

Total cellular RNA was isolated using acid-guanidinium phenol/chloroform extraction as described elsewhere (Chomczynski and Sacchi, Anal Biochem. 162:156-9, (1987)). Equal amounts of RNA (15 μg per lane) were fractionated on 1% denaturing formaldehyde-agarose gels. RNA was transferred to Hybond nylon membranes (Amersham, Piscataway, N.J.), UV cross-linked, and then hybridized with cDNA probes specific for PSA or GAPDH labeled with α³²P-dCTP using a RadPrime labeling kit (Invitrogen). Autoradiography was performed at −80° C. using an intensifier screen (Kodak).

Western Blot Analysis

Cells were harvested directly in a loading buffer containing 65 mM Tris-HCl (pH 7.0), 2% (w/v) SDS, 5% β-mercaptoethanol, 10% (v/v) glycerol and 0.5% (w/v) bromophenol blue. The protein concentration in each sample was determined. Equal amounts of protein (typically 30 μg per lane) were resolved in 10% NuPAGE gels (Invitrogen) and transferred to nitrocellulose membranes that were then blocked from non-specific binding. The membranes were incubated with antibodies specific for AR (N-20; Santa Cruz Biotechnology, Santa Cruz, Calif.), ERK-2 (D-2; Santa Cruz Biotechnology), or Gal4 (RK5C1; Santa Cruz Biotechnology) at a final concentration of 100 ng/mL. The membranes were then washed and incubated with an appropriate secondary antibody conjugated to horseradish peroxidase (Santa Cruz Biotechnology) that was diluted 1:10,000. The membranes were immersed in chemiluminescence reagents (Pierce, Rockford, Ill.) and exposed to Kodak X-Omat Blue XB-1 film for signal detection.

Cell Viability Assays

C4-2 cells were seeded at a density of 3000 cells per well of a 96-well plate in RPMI 1640 containing 5% CSS. After 24 hours, the medium was replaced with RPMI 1640 containing 5% CSS and 10 μM bicalutamide or ethanol (vehicle control). Following 0, 24, 48, and 72 hours, the number of viable cells was determined by MTS reduction (Cell Titer 96 AQueous One, Promega), which was measured at 490 nm. The data are presented as the mean±S.E.M. of quadruplicate experiments.

### Example 2

### AR is Constitutively Active in ADI Prostate Cancer Cells

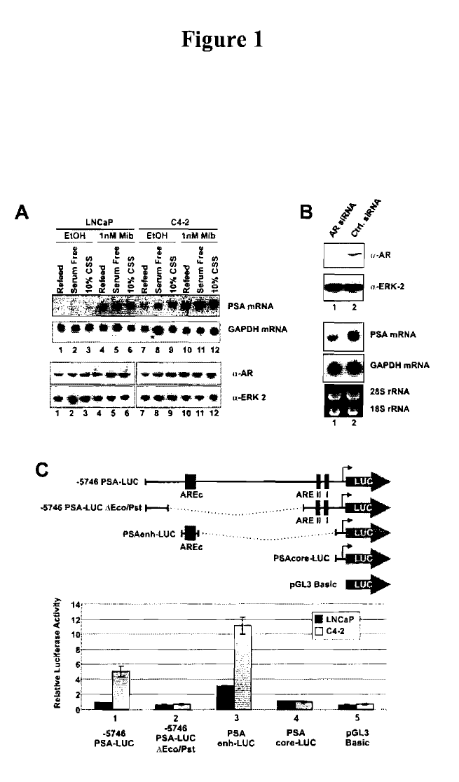

The isogenic LNCaP/C4-2 cell model of prostate cancer progression was used to study regulated and de-regulated mechanisms of AR activation. C4-2 cells are ADI and were derived from androgen-dependent LNCaP cells through serial passage as xenografts in castrated athymic male mice (Wu et al., Int J Cancer, 57:406-12 (1994)). To characterize endogenous AR activity in this model of prostate cancer progression, LNCaP and C4-2 cells were cultured in the presence or absence of 1 nM mibolerone, a synthetic androgen. In the absence of androgen, LNCaP cells expressed very low mRNA levels of the AR target gene, PSA, regardless of whether they were cultured in the absence of serum or in the presence of steroid-depleted serum (FIG. 1A, lanes 2 and 3). Androgen treatment induced high levels of PSA mRNA expression in LNCaP cells (FIG. 1A, lanes 5 and 6). In C4-2 cells, constitutive PSA mRNA expression was observed, regardless of whether cells were cultured in the absence of serum or in the presence of steroid-depleted serum (FIG. 1A, lanes 8 and 9). PSA expression in C4-2 cells was further increased by androgen treatment (FIG. 1A, lanes 11 and 12).

The possible role of autocrine factors in regulating PSA expression (Heinlein and Chang, Endocr Rev., 25:276-308 (2004)) was also tested. A six hour re-feeding regimen, intended to minimize the accumulation of autocrine factors, did not abolish the androgen-dependent or -independent expression of PSA mRNA in LNCaP or C4-2 cells (FIG. 1A, lanes 1, 4, 7, and 10). Constitutive PSA expression in C4-2 cells was not the result of higher AR levels in this cell line compared to the LNCaP cell line (FIG. 1A).

Small interfering RNA (siRNA) was used to test the AR-dependence of androgen independent PSA expression in C4-2 cells. A decrease in expression of PSA mRNA was observed in C4-2 cells transfected with AR-targeted siRNA as compared to the expression of PSA mRNA in cells transfected with non-targeted siRNA (FIG. 1B). These data indicate that the AR is aberrantly active in the absence of androgens in C4-2 cells, and PSA expression serves as a sensitive indicator of this activity.

To identify regions of the PSA promoter that mediate constitutive AR activity in C4-2 cells, LNCaP and C4-2 cells were transfected with an expression construct regulated by a 5.8 kb PSA promoter fragment (−5746 PSA-LUC). The activity of the −5746 PSA-LUC construct was assessed in the absence of serum and androgens. Transfection efficiencies were similar for the LNCaP and C4-2 cell lines. The activity of the −5746 PSA-LUC construct was higher in the C4-2 cells than in the LNCaP cells (FIG. 1C). The upstream PSA enhancer core (AREc) mediated elevated androgen-independent PSA promoter activity in C4-2 cells, and deletion constructs lacking this element did not display detectable activity (FIG. 1C). These findings indicate that the enhancer core of the PSA promoter contains elements that are regulated by constitutively active AR in C4-2 cells.

### Example 3

### An AR-Gal4 Fusion Strategy Enables Functional Studies of the AR

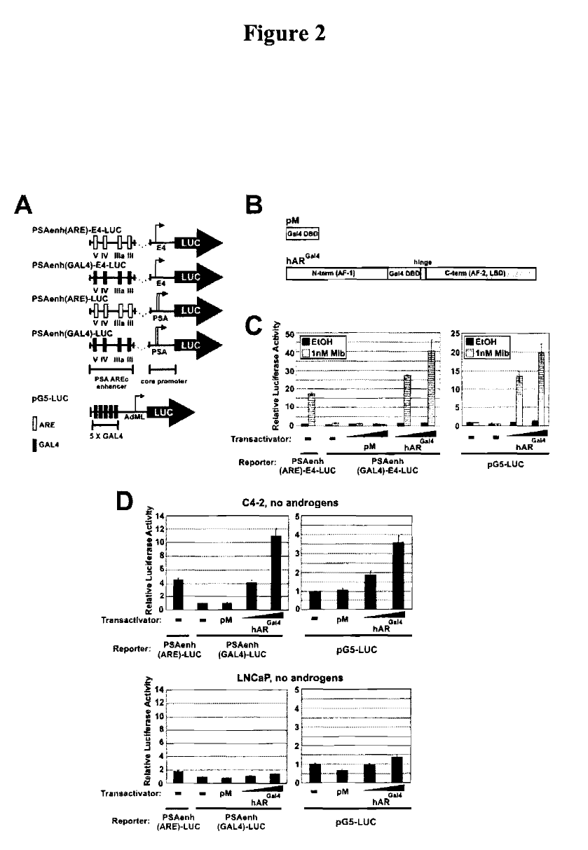

Ligand-bound AR recruits coregulatory proteins to genomic androgen response elements (AREs) in the promoter and enhancer regions of target genes (Horlein et al., Nature, 377:397-404 (1995)). To study AR structure/function relationships in LNCaP and C4-2 cells, an AR-Gal4 fusion strategy was developed. This strategy was based on the premise that swapping AREs in PSA enhancer-based reporter constructs with binding sites for the Gal4 yeast transcription factor would render these constructs responsive to AR^{Gal4}, a hybrid protein wherein the AR zinc-finger DBD was swapped with the Gal4 zinc-finger DBD (FIGS. 2A and 2B). An ARE-driven promoter, PSAenh(ARE)E4-LUC, displayed robust activation in response to 1 nM mibolerone (FIG. 2C), and replacement of the four AREs with GAL4 binding sites completely abolished this androgen response (FIG. 2C). Co-transfection with an expression construct encoding the Gal4 DBD alone failed to restore androgen responsiveness (FIG. 2C). However, co-transfection with hAR^{Gal4} restored a robust androgen response (FIG. 2C). hAR^{Gal4} was also able to activate pG5-LUC, a reporter construct containing five tandem GAL4 binding sites upstream of the adenovirus major late core promoter, only in the presence of androgen (FIG. 2C).

To test whether this system was a sensitive measure of constitutive AR activity in C4-2 cells, the AREs in PSAenh(ARE)-LUC were replaced with GAL4 binding sites (FIG. 2A). Under androgen-free conditions, replacement of AREs with GAL4 binding sites significantly inhibited promoter activity in C4-2 cells, but not in LNCaP cells (FIG. 2D). Co-transfection with hAR^{Gal4} resulted in robust androgen-independent activation of PSAenh(GAL4)-LUC in C4-2 cells, but not LNCAP cells (FIG. 2D). To determine whether these observations were restricted to the PSA gene, or whether they represented a more general indication of AR activity in these cells, the ability of hAR^{Gal4} to activate pG5-LUC in an androgen independent manner was tested. Indeed, hAR^{Gal4} was able to activate pG5-LUC in C4-2 but not LNCaP cells in the absence of androgens (FIG. 2D). These results indicate that the Gal4-based system represents a powerful tool for functional studies of the AR.

### Example 4

### Androgen-Independent hAR^{Gal4} Activity is AF-2 Independent

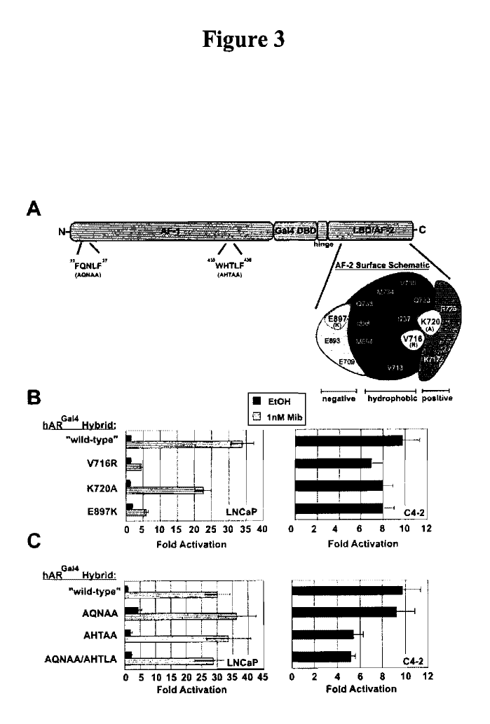

The Gal4-based system was used to investigate the role of the AF-2 domain in ligand-dependent and ligand-independent AR activation in LNCaP and C4-2 cells. Specific point mutations were generated in the AF-2 domain of the AR polypeptide (FIG. 3A). The mutations V716R, K720A, and E897K in the hAR^{Gal4} polypeptide compromised androgen-induced activation of the PSAenh(GAL4)-LUC construct as compared to the activation observed using the wild-type hAR^{Gal4} polypeptide in LNCaP cells (FIG. 3B). Similar results were observed when these experiments were repeated using the pG5-LUC reporter construct. In contrast, the AF-2 mutations did not have a significant effect on androgen-independent hAR^{Gal4} activity in C4-2 cells (FIG. 3B). These results indicate that androgen-dependent AR activity in LNCaP cells is dependent on AF-2, whereas androgen-independent AR activity in C4-2 cells is AF-2 independent.

The AF-2 domain of the AR can mediate interaction with the amino-terminus of the AR. Mutations were introduced in the amino-terminal ²³FQNLF²⁷ (SEQ ID NO:22) and ⁴³⁵WHTLF⁴³⁹ (SEQ ID NO:9) motifs of the AR polypeptide, which abolish the AR amino- and carboxy-terminal interaction (He et al., J Biol Chem., 277:25631-9 (2002); He et al., J Biol Chem., 275:22986-94 (2000)). The ²³AQNAA²⁷ (SEQ ID NO:2) mutation, alone or in combination with the ⁴³⁵AHTAA⁴³⁹ (SEQ ID NO:3) mutation, did not inhibit hAR^{Gal4} activity in LNCAP cells (FIG. 3C). Similarly, the ²³AQNAA²⁷ (SEQ ID NO:2) mutation had no effect on androgen independent hAR^{Gal4} activity in C4-2 cells (FIG. 3C). Surprisingly, the ⁴³⁵AHTAA⁴³⁹ (SEQ ID NO:3) mutation, alone or in conjunction with the ²³AQNAA²⁷ (SEQ ID NO:2) mutation, significantly decreased androgen-independent activation of PSAenh(GAL4)-LUC by hAR^{Gal4} in C4-2 cells (FIG. 3C). Taken together, these results demonstrate that the AR N-C interaction is dispensable for AR activity in LNCaP cells. This suggests that in LNCAP cells, the AR AF-2 domain primarily interacts with key coregulatory proteins.

### Example 5

### Constitutively Active AR NTD Motifs Mediate Ligand-Independent AR Activity

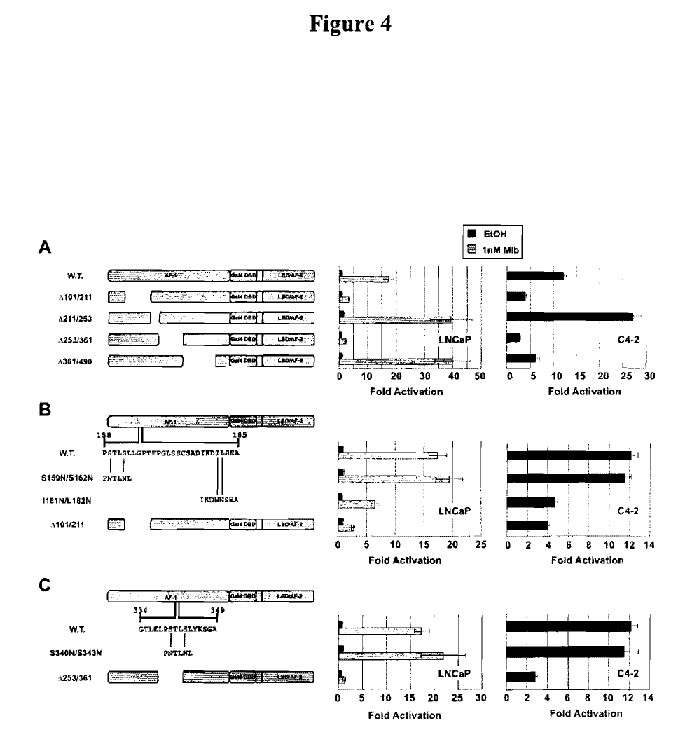

To identify putative targets for AR inhibition in ADI prostate cancer cells, the effects of NTD truncations on ligand-dependent and ligand-independent AR activity were tested in LNCaP and C4-2 cells, respectively. Deletion of amino acids 101-211 or 253-361 impaired both ligand-dependent and ligand-independent AR activity (FIG. 4A). Conversely, an NTD deletion of amino acids 211-253 resulted in a two-fold increase in both ligand-dependent and ligand-independent AR activity (FIG. 4A). Surprisingly, deletion of AR amino acids 361-490 resulted in a two-fold increase in ligand-dependent AR activity and a two-fold decrease in ligand-independent AR activity (FIG. 4A).

An I18IN/L182N double mutation significantly inhibited both ligand-dependent and ligand-independent AR^{Gal4} activity in LNCaP and C4-2 cells, respectively (FIG. 4B). This finding demonstrates the importance of I181 and L182 for ligand-dependent AR activity in prostate cancer cells, and also reveals the importance of these residues for ligand-independent AR activity in ADI prostate cancer cells. A neighboring motif within this region, ¹⁵⁸PSTLSL¹⁶³ (SEQ ID NO:23), is reported to mediate binding to the RAP74 subunit of the basal transcription factor, TFIIF (Reid et al., J Biol Chem., 277:41247-53 (2002)). A S159N/S162N mutation, which disrupts the AR-RAP74 interaction (Reid et al., J Biol Chem., 277:41247-53 (2002)), was observed to have no effect on either ligand-dependent or ligand-independent AR activity. These results suggest that I181 and L182 are the primary components of the AR transcriptional activation domain encompassed by amino acids 101-211.

A second PSTLSL (SEQ ID NO:23) motif, located within amino acids 339-344 of the AR, is also reported to mediate binding to the RAP74 subunit of TFIIF (Reid et al., J Biol Chem., 277:41247-53 (2002)). It was determined whether loss of this motif was responsible for the transcriptional defect observed in the 253/361 AR deletion. A S340N/S343N mutation, which disrupts the AR-RAP74 interaction (Reid et al., J Biol Chem., 277:41247-53 (2002)), did not abolish either ligand-dependent or ligand-independent AR activity (FIG. 4C). These data indicate that the ³³⁹PSTLSL³⁴⁴ (SEQ ID NO:23) motif, similar to the ¹⁵⁸PSTLSL¹⁶³ (SEQ ID NO:23) motif, does not play a role in directing AR transcriptional activity in prostate cancer cells.

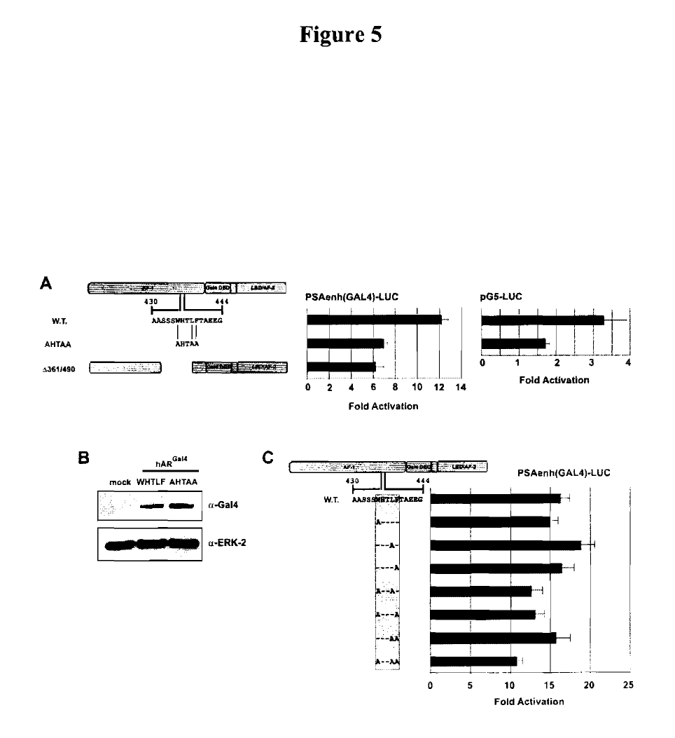

Characterization of AR AF-2 dependence in ADI prostate cancer cells demonstrated that the NTD ⁴³⁵WHTLF⁴³⁹ (SEQ ID NO:9) motif was important for ligand-independent AR activity, but dispensable for ligand-dependent AR activity (FIG. 3C). Similarly, a deletion encompassing 361/490 inhibited ligand-independent AR activity while resulting in a two-fold increase in ligand-dependent AR activity (FIG. 4A). A similar two-fold reduction in ligand-independent AR activity was observed when the activities of the ⁴³⁵AHTAA⁴³⁹ (SEQ ID NO:3) triple mutant and Δ361/490 deletion versions of the AR were compared directly (FIG. 5A). A two-fold reduction in ligand-independent activity was also observed for ⁴³⁵AHTAA⁴³⁹ (SEQ ID NO:3) when pG5-LUC was used as a reporter for these assays (FIG. 5A). Differential ligand-independent activity was not due to differential expression of the wild-type and ⁴³⁵AHTAA⁴³⁹ (SEQ ID NO:3) versions of AR^{Gal4} in these cells (FIG. 5B). Testing of single and double alanine substitutions within the ⁴³⁵WHTLF⁴³⁹ (SEQ ID NO:9) core indicated that all single substitutions as well as the ⁴³⁵WHTAA⁴³⁹ (SEQ ID NO:24) double substitution had no effect (FIG. 5C). Conversely, the ⁴³⁵AHTAF⁴³⁹ (SEQ ID NO:25) and ⁴³⁵AHTLA⁴³⁹ (SEQ ID NO:26) double substitutions had an effect intermediate to the ⁴³⁵AHTAA⁴³⁹ (SEQ ID NO:3) triple substitution (FIG. 5C). Taken together, these data indicate that ⁴³⁵WHTLF⁴³⁹ (SEQ ID NO:9) is the major component of the AR transcriptional activation domain encompassed by amino acids 361-490. Furthermore, these results demonstrate that this domain plays a novel role in mediating ligand-independent AR activity in ADI prostate cancer cells.

### Example 6

### Ligand-Independent AR Activity is Cross-Resistant to Antiandrogens

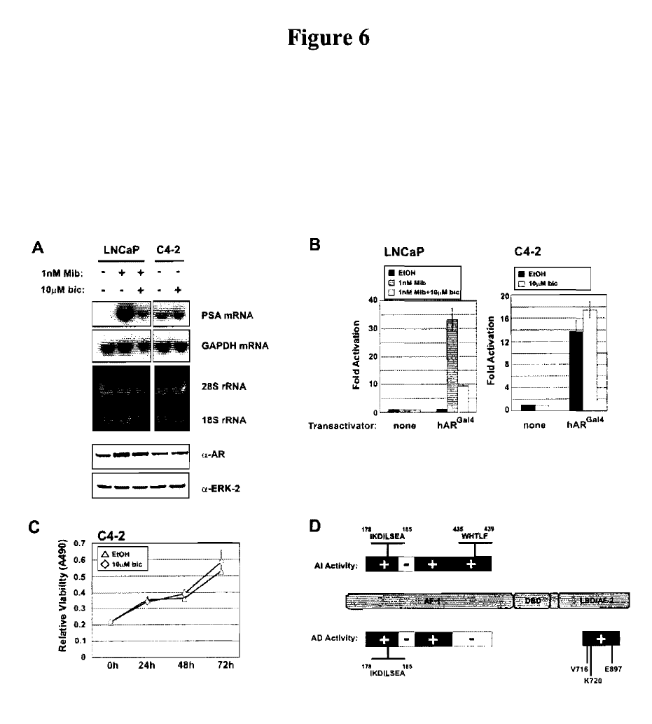

The effect of bicalutamide on androgen-dependent and -independent AR activity was determined in LNCaP and C4-2 cells, respectively. In LNCaP cells, 10 μM bicalutamide inhibited androgen-dependent induction of PSA expression (FIG. 6A). In C4-2 cells, constitutive PSA expression was unaffected, or even slightly increased in the presence of 10 μM bicalutamide (FIG. 6A). In LNCaP cells, bicalutamide inhibited the androgen-induced ability of hAR^{Gal4} to activate the PSAenh(GAL4)-LUC reporter (FIG. 6B). Conversely, in C4-2 cells, bicalutamide had no effect on, or slightly increased, androgen-independent hAR^{Gal4} activity (FIG. 6B). Moreover, bicalutamide was unable to affect the overall viability of C4-2 cells growing in androgen-depleted medium (FIG. 6C). These data reveal that constitutive AR activity in C4-2 cells is resistant to ligand ablation as well as direct antagonism.

### Example 7

### WxxLF Mediates Ligand-Independent AR Activity

The role of the NH₂-terminal domain (NTD) transactivation unit 5 (TAU5) domain in mediating AR transcriptional activity was investigated in cell-based models of prostate cancer progression.

Cell Lines and Culture Conditions

Androgen dependent LNCaP and ADI 22Rv1 cell lines were purchased from the American Type Culture Collection (ATCC). The ADI LNCaP-derived C4-2 cell line was purchased from UroCor (Oklahoma City, Okla.). LNCaP, C4-2, and 22Rv1 cells were grown in RPMI 1640 medium supplemented with 10% fetal bovine serum (FBS). For androgen response experiments, cells were grown in serum free medium for 48 hours. Growth medium was aspirated and replaced with serum free medium containing 1 nM mibolerone (BIOMOL, Plymouth Meeting, Pa.), 10 μM bicalutamide (Casodex®, AstraZeneca), ethanol (vehicle control), or combinations of these compounds. Cells were cultured for an additional 24 hours and harvested in specific lysis buffers. For 22Rv1 siRNA-transfection experiments, culture medium was replaced 24 hours post-transfection with serum-free, phenol red-free RPMI. After 48 hours of growth, cells were harvested in specific lysis buffers.

Plasmid Constructs

MMTV-Luc was provided by Dr. Frank Claessens (Catholic University of Leuven, Belgium). The p5HBhAR-A plasmid, encoding wild-type human (h)AR (Dehm and Tindall, J Biol Chem, 281:27882-27893 (2006)) was rendered resistant to an AR-targeted small-interference RNA (siRNA) by introducing three silent mutations at AR codons 314, 315, and 316 (GGGGGCTAT, mutant bases underlined) using a QuikChange (Stratagene) site-directed mutagenesis kit (referred to as hAR^{sr}). SV40-Renilla luciferase and pG5-LUC reporter vectors were purchased from Promega and Clontech, respectively. The plasmids PSAenh(ARE)-LUC, PSAenh(GAL4)-LUC, hAR^{Gal4}, hAR^{Gal4} ΔTAU5, and NTD^{Gal4} are described elsewhere (Dehm and Tindall, J Biol Chem, 281:27882-27893 (2006); Debes et al., Cancer Res, 65:5965-5973 (2005)). Point mutant versions of hAR^{sr} and hAR^{Gal4} were generated using QuikChange site-directed mutagenesis coupled with appropriately designed mutagenic primers. Deletion mutant versions of hAR^{Gal4} and NTD^{Gal4} were generated using QuikChange mutagenesis to create BssHII sites flanking the desired deletion, digesting with BssHII, and re-ligating the plasmid. hAR^{Gal4} ΔTAU5/whtlf+, hAR^{Gal4} ΔTAU5/ahtaa+, hAR^{Gal4} ΔTAU5/2Xwhtlf+, and hAR^{Gal4} ΔTAU5/3Xwhtlf+ were generated by inserting synthetic double-stranded cassettes with flanking BssHII sites into BssHII-digested hAR^{Gal4} ΔTAU5. Fusions between the Gal4 DBD (pM, Clontech) and WHTLF (SEQ ID NO:9)- or AHTAA (SEQ ID NO:3)-containing peptides were constructed using the same strategy. Sequences of cassettes and primers used for mutagenesis reactions are presented in Table 1. All deletion and point-mutant constructs were sequenced to verify their integrity and were tested for expression via transient transfection and Western blot with anti-AR and anti-Gal4 polypeptide antibodies.

TABLE 1
Sequences of primers and cassettes

| | | |
|---|---|---|
| Description | Sequence | SEQ ID NO: |

| | | |
|---|---|---|
| Primers for generating ΔTAU5 | | |
| version of AR^{Gal4}: | | |
| BssHII @ 361: | | |
| 1. AR_361_BssFWD | 5′-CGAGGCAGCTGCGTACCAGgcgCGCGA | 29 |
| | CTACTACAACTTTCCACTGG | |
| 2. AR_361_BssREV | 5′-CCAGTGGAAAGTTGTAGTAGTCGCGcgc | 30 |
| | CTGGTACGCAGCTGCCTCG | |
| BssHII @ 490: | | |
| 1. AR_490_BssFWD | 5′-CGGCCCCCTCAGGGGCTGGCGcGCCA | 31 |
| | GGAAAGCGACTTCACC | |
| 2. AR_490_BssREV | 5′-GGTGAAGTCGCTTTCCTGGCgCGCCAG | 32 |
| | CCCCTGAGGGGGCCG | |
| Primers for generating Δ392- | | |
| 420 version of AR^{Gal4}: | | |
| BssHII @ 392: | | |
| 1. AR_392_BssFWD | 5′-CTGGAGAACCCGCTGGcgcgCGGCAGC | 33 |
| | GCCTGGGCGGC | |
| 2. AR_392_BssREV | 5′-GCCGCCCAGGCGCTGCCGcgcgCCAGCG | 34 |
| | GGTTCTCCAG | |
| BssHII @ 420: | | |
| 1. AR_420_BssFWD | 5′-CATGGCGCGGGTGCAGCGcGcCCCGGT | 35 |
| | TCTGGGTCAC | |
| 2. AR_420_BssREV | 5′-GTGACCCAGAACCGGGgCgCGCTGCAC | 36 |
| | CCGCGCCATG | |
| Primers for generating Δ420- | | |
| 449 version of AR^{Gal4}: | | |
| BssHII @ 420: | | |
| 3. AR_420_BssFWD | 5′-CATGGCGCGGGTGCAGCGcGcCCCG | 35 |
| | GTTCTGGGTCAC | |
| 4. AR_420_BssREV | 5′-GTGACCCAGAACCGGGgCgCGCTGCAC | 36 |
| | CCGCGCCATG | |
| BssHII @ 449: | | |
| 1. AR_449_BssFWD | 5′-GGCCAGTTGTATGGA+EE,uns gCGcGcGGTGGT | 37 |
| | GGTGGGGGTGG | |
| 2. AR_449_BssREV | 5′-CCACCCCCACCACCACCgCgCGcTCCAT | 38 |
| | ACAACTGGCC | |
| Cassette for 1XWHTLF insert | | |
| in ΔTAU5 AR^{Gal4}: | | |
| 426-450wtBssFW | 5′-CgCgCTCAGCCGCCGCTTCCTCATCCTG | 39 |
| | GCACACTCTCTTCACAGCCGAAGAAGG | |
| | CCAGTTGTATGGAg | |
| 426-450wtBssRV | 5′-CgCGcTCCATACAACTGGCCTTCTTCGG | 40 |
| | CTGTGAAGAGAGTGTGCCAGGATGAG | |
| | GAAGCGGCGGCTGAG | |
| Cassette for 2XWHTLF insert | | |
| in ΔTAU5 AR^{Gal4}: | | |
| 2X WHTLF-FW | 5′-cgcgcTCAGCCGCCGCTTCCTCATCCTGG | 41 |
| | CACACTCTCTTCACAGCCGAAGAAGGC | |
| | CAGTTGTATGGATCAGCCGCCGCTTCC | |
| | TCATCCTGGCACACTCTCTTCACAGCC | |
| | GAAGAAGGCCAGTTGTATGGAg | |
| 2X WHTLF-RV | 5′-cgcgcTCCATACAACTGGCCTTCTTCGGC | 42 |
| | TGTGAAGAGAGTGTGCCAGGATGAGG | |
| | AAGCGGCGGCTGATCCATACAACTGGC | |
| | CTTCTTCGGCTGTGAAGAGAGTGTGCC | |
| | AGGATGAGGAAGCGGCGGCTGAg | |
| Cassette for 2XWHTLF insert | | |
| in ΔTAU5 AR^{Gal4}: | | |
| 3X WHTLF-FW | 5′-cgcgcTCAGCCGCCGCTTCCTCATCCTGG | 43 |
| | CACACTCTCTTCACAGCCGAAGAAGGC | |
| | CAGTTGTATGGATCAGCCGCCGCTTCC | |
| | TCATCCTGGCACACTCTCTTCACAGCC | |
| | GAAGAAGGCCAGTTGTATGGATCAGC | |
| | CGCCGCTTCCTCATCCTGGCACACTCT | |
| | CTTCACAGCCGAAGAAGGCCAGTTGTA | |
| | TGGAg | |
| 3X WHTLF-RV | 5′-cgcgcTCCATACAACTGGCCTTCTTCGGC | 44 |
| | TGTGAAGAGAGTGTGCCAGGATGAGG | |
| | AAGCGGCGGCTGATCCATACAACTGGC | |
| | CTTCTTCGGCTGTGAAGAGAGTGTGCC | |
| | AGGATGAGGAAGCGGCGGCTGATCCA | |
| | TACAACTGGCCTTCTTCGGCTGTGAAG | |
| | AGAGTGTGCCAGGATGAGGAAGCGGC | |
| | GGCTGAg | |
| Cassette for 1XAHTAA insert | | |
| in ΔTAU5 AR^{Gal4}: | | |
| 426-450AAABssFW | 5′-CgCgCTCAGCCGCCGCTTCCTCATCCgc | 45 |
| | GCACACTgcCgcCACAGCCGAAGAAGGC | |
| | CAGTTGTATGGAg | |
| 426-450AAABssRV | 5′-CgCGcTCCATACAACTGGCCTTCTTCGG | 46 |
| | CTGTGgcGgcAGTGTGCgcGGATGAGGA | |
| | AGCGGCGGCTGAG | |

Transient Transfections

For siRNA transfections, 3×10⁶ 22Rv1 cells from exponentially growing cultures were mixed with 80 pmoles of AR-targeted siRNA (ARsiRNA1 sense: 5′-CAAGGGAG GUUACACCAAAUU (SEQ ID NO:27); ARsiRNA2 sense: 5′-GAAAUGAUUGCACU AUUGAUU (SEQ ID NO:28)) or a non-targeted control siRNA (Dharmacon). The cell/siRNA mixture was transferred to electroporation cuvettes with a 4 mm gap-width (BTX) and was subjected to a 350 V electrical pulse for 10 ms using a BTX ElectroSquare Electroporator. Following a 15 minute recovery period, the cells were seeded in RPMI 1640+5% charcoal-stripped, steroid-depleted serum (CSS).

For siRNA transfections coupled with rescue by hAR^{sr}, 1×10⁵ cells were seeded in 24-well dishes and transfected the following day with 375 ng of a MMTV-LUC plasmid, 125 ng of SV40-Renilla luciferase reporter vector, 10 pmoles of ARsiRNA1 (or control siRNA), and 11.75 ng of AR^{sr}, in complex with 2 μL of Lipofectamine 2000 (Invitrogen) according to the manufacturer's protocol. Following 6 hours of transfection, the medium was aspirated and replaced with the appropriate medium containing 5% CSS. In addition to the Lipofectamine 2000-based protocol, electroporation was also employed as a mode of transfection for siRNA rescue experiments with hAR^{sr}. For this approach, amounts of plasmids were increased by a factor of 24 from the Lipofectamine 2000-based protocol. For a standard electroporation, 3×10⁶ cells from exponentially growing cultures were suspended in 350 μL of RPMI+5% CSS and mixed with 50 μL of a DNA/siRNA mixture containing 9 μg of MMTV-LUC plasmid, 3 μg of SV40-Renilla luciferase reporter vector, 80 pmoles of ARsiRNA1 (or control siRNA), and 282 ng of hAR^{sr}. The cell/DNA mixture was transferred to electroporation cuvettes with a 4 mm gap-width (BTX) and was subjected to a 305 V (LNCaP, C4-2) or a 350 V (22Rv1) electrical pulse for 10 ms using a BTX ElectroSquare Electroporator. Following a 15 minute recovery period, cells were seeded in RPMI 1640+5% CSS.

hAR^{Gal4}-based tethering assays were performed in LNCaP and C4-2 cells as described elsewhere (Dehm and Tindall, J Biol Chem, 281:27882-27893 (2006)) with minor modification. Briefly, 1×10⁵ cells were seeded in 24-well dishes and transfected the following day using 2.5 μL of Superfect (Qiagen) mixed with 375 ng of either PSAenh(ARE)-LUC or PSAenh(GAL4)-LUC as a reporter construct, 125 ng of SV40-Renilla luciferase reporter vector, and 23.4 ng of hAR^{Gal4}. Transfections were performed in the presence of RPMI+5% CSS. In addition to the Superfect-based protocol, electroporation was also employed as a mode of transfection for hAR^{Gal4}-based tethering assays. For this approach, amounts of plasmids were increased by a factor of 24 from the Superfect-based protocol. For a standard electroporation, 3×10⁶ cells from exponentially growing cultures were suspended in 350 μL of RPMI+5% CSS and mixed with 50 μL of a DNA mixture containing 9 μg of a promoter-LUC reporter, 3 μg of SV40-Renilla luciferase reporter vector, and 562 ng of hAR^{Gal4}. The cell/DNA mixture was transferred to electroporation cuvettes with a 4 mm gap-width (BTX) and was subjected to a 305 V electrical pulse for 10 ms using a BTX ElectroSquare Electroporator. Following a 15 minute recovery period, the cells were seeded in RPMI 1640+5% CSS.

Overall transfection efficiency was optimized for each experiment by transfecting cells with green fluorescent protein (GFP), and assessing the number of fluorescent cells 24 hours post-transfection. Transfection efficiencies consistently ranged from 60-80%. For all reporter-based experiments, medium was aspirated 24 hours post-transfection and replaced with serum-free, phenol red-free RPMI 1640 containing 1 nM mibolerone, 2 pM-1 nM DHT (Sigma Aldrich), or ethanol (vehicle control). Cells were harvested after an additional 24 hours and processed in a lysis buffer provided with a Dual Luciferase Assay Kit (Promega). Activities of the firefly and Renilla luciferase reporters were assayed in 96-well plates using a Dual Luciferase Assay Kit and detected with a Molecular Devices LMax luminometer. Transfection efficiency was addressed by dividing firefly luciferase activity by Renilla luciferase activity of samples cultured in the absence of androgens. Data presented represent the mean±S.E.M. from at least three independent experiments, each performed in duplicate.

RNA Extraction and Northern Blot Analysis

Total cellular RNA was isolated via acid-guanidinium phenol/chloroform extraction as described elsewhere (Chomczynski and Sacchi, Anal Biochem, 162:156-159 (1987)). Equal amounts of RNA (15 μg per lane) were fractionated on 1% denaturing formaldehyde-agarose gels. RNA was transferred to Hybond nylon membranes (Amersham), UV cross-linked, and hybridized with cDNA probes specific for PSA or GAPDH labeled with α³²P-dCTP using a RadPrime labeling kit (Invitrogen). Autoradiography was performed at −80° C. using an intensifier screen (Kodak).

Western Blot Analysis

Cells were harvested directly in a loading buffer containing 65 mM Tris-HCI (pH 7.0), 2% (w/v) SDS, 5% β-mercaptoethanol, 10% (v/v) glycerol, and 0.5% (w/v) bromophenol blue. Protein concentrations were determined using a kit based on a modified Lowry assay (BioRad RC/DC Assay). Equal amounts (typically 30 μg per lane) of protein were resolved in 10% SDS-polyacrylamide gels (Invitrogen), followed by transfer to nitrocellulose and membrane blocking. Blots were incubated with antibodies specific for AR (Santa Cruz, C-19 or N-20), ERK-2 (Santa Cruz, D-2), or Gal4 (Santa Cruz, RK5C1) at a final concentration of 100 ng/mL. The blots were then washed and probed with an appropriate secondary antibody conjugated to horseradish peroxidase (Santa Cruz) and diluted 1:10,000. Membranes were immersed in chemiluminescence reagents (Pierce) and exposed to Kodak XAR film for signal detection.

Results

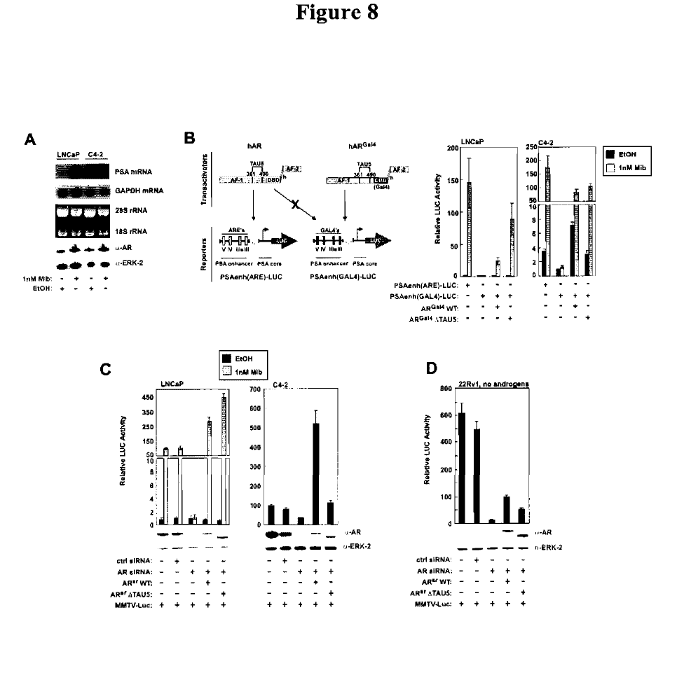

The LNCaP/C4-2 model of prostate cancer progression was used to study the mechanisms of AR transcriptional regulation in androgen-dependent and ADI prostate cancer cells (Dehm and Tindall, J Biol Chem, 281:27882-27893 (2006); Zegarra-Moro et al., Cancer Research, 62:1008-1013, (2002)). The ADI C4-2 cell line was derived from the androgen-dependent LNCaP cell line through serial xenografting in castrated hosts (Wu et al., Int J Cancer, 57:406-412 (1994)). A key feature of the model is that LNCaP cells display ligand-dependent mRNA expression of the AR-regulated prostate specific antigen (PSA) gene, whereas C4-2 cells display constitutive PSA mRNA expression that can be further enhanced by androgens (FIG. 8A). Experiments employing siRNA-mediated AR knock-down and chromatin immunoprecipitation (ChIP) demonstrated that constitutive PSA expression is due to constitutive, ligand-independent AR transcriptional activity on the PSA promoter in C4-2 cells (Yeung et al., J Biol Chem, 275:40846-40855 (2000); Jia et al., Mol Cell Biol, 26:7331-7341 (2006)). A promoter tethering system that can be used to study structural and functional domains required by the AR to mediate ligand-independent activity is described elsewhere (Dehm and Tindall, J Biol Chem, 281:27882-27893 (2006)). The strategy involves swapping androgen response elements (AREs) in a PSA-based promoter reporter construct (PSAenh(ARE)-LUC) with binding sites for the yeast Gal4 transcription factor (PSAenh(GAL4)-LUC) and concurrently swapping the zinc-finger AR DBD with the zinc-finger DBD of Gal4 to create chimeric hAR^{Gal4} (FIG. 8B, left panel). The constructs provide a sensitive monitoring system of AR activity in AR-expressing prostate cancer cells, which functions independent of the endogenous AR. Using this system, it was demonstrated that constitutive AR activity is independent of the AR AF-2 domain in C4-2 cells (Dehm and Tindall, J Biol Chem, 281:27882-27893 (2006)). Based on these observations, attention was focused on transcriptional activation domains within the unstructured AR NTD that may be responsible for mediating ligand-independent AR activity in ADI prostate cancer cells.

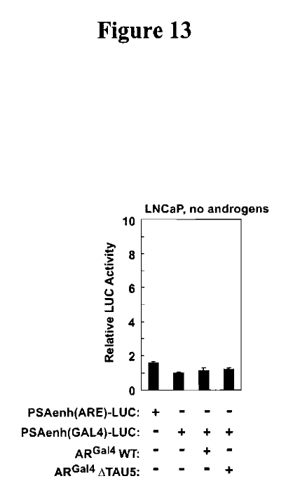

A region of the AR NTD encompassing amino acids 361-490 was termed TAU5 by virtue of its importance for full ligand-induced activity of ectopic AR in AR-null HeLa cells (Jenster et al., J Biol Chem, 270:7341-7346 (1995)). To assess the role of TAU5 in mediating AR activity, wild-type and TAU5-deleted (ΔTAU5) versions of hAR^{Gal4} were tethered to PSAenh(GAL4)-LUC. GAL4 substitution of AREs in the PSA-based reporter construct completely abolished androgen-induced promoter activity in both LNCaP and C4-2 cells (FIG. 8B). Importantly, GAL4 substitution of AREs also inhibited androgen-independent PSA promoter activity 4-fold, only in C4-2 cells (FIG. 8B). In LNCAP cells, hAR^{Gal4} ΔTAU5 displayed over two-fold higher androgen-induced transcriptional activity than wild-type hAR^{Gal4} (FIGS. 8B and 13). Conversely, ΔTAU5 hAR^{Gal4} displayed over two-fold lower androgen-independent transcriptional activity when compared to wild-type hAR^{Gal4} in C4-2 cells (FIG. 8B). Despite this observation, deletion of TAU5 had no effect on the full level of ligand-induced hAR^{Gal4} activity in C4-2 cells (FIG. 8B). These results demonstrate that TAU5 deletion selectively impairs ligand-independent hAR^{Gal4} transcriptional activity in ADI C4-2 cells.

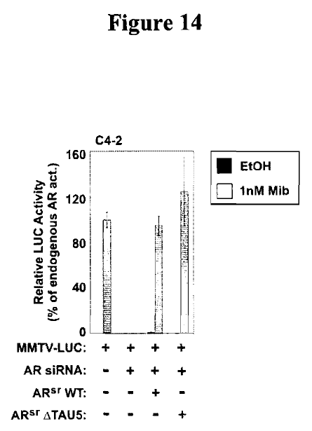

To ensure that these findings did not represent an artifact of chimeric hAR^{Gal4}, and were not restricted to a PSA-based reporter construct, an AR replacement strategy was employed. Wild-type and ΔTAU5 forms of wild-type hAR that were resistant to an AR-targeted siRNA (hARs) were generated and tested their ability to stimulate the MMTV promoter in LNCaP and C4-2 cells (FIG. 8C). AR-targeted siRNA completely attenuated androgen-responsiveness of the MMTV promoter in LNCaP cells, but had no effect on ligand-independent MMTV promoter activity (FIG. 8C). Consistent with this finding, expression of hAR^{sr} restored androgen-dependent MMTV activity in LNCaP cells (FIG. 8C). Conversely, AR-targeted siRNA inhibited ligand-independent MMTV promoter activity by over 60% in C4-2 cells, which was restored upon expression of hARSr (FIG. 8C). In addition, as observed in Gal4 tethering experiments (FIG. 8B), TAU5 deletion impaired ligand-independent, but not ligand dependent hAR^{sr} activity in C4-2 cells (FIGS. 8C and 14).

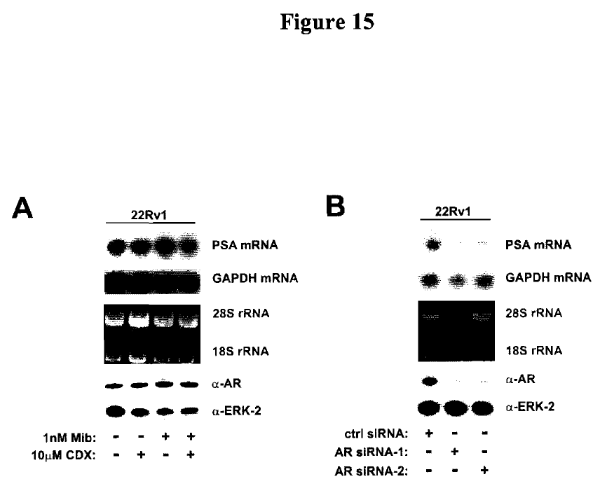

To examine whether TAU5 plays a role in mediating aberrant AR activity in other, non-LNCaP-based models of ADI prostate cancer, the ADI 22Rv1 prostate cancer cell line was employed. The ADI 22Rv1 cell line was derived from a CWR22 prostate cancer xenograft that relapsed following host castration-induced regression (Srainkoski et al., In Vitro Cell Dev Biol Anim, 35:403-409 (1999)). In contrast to the T877A mutant AR expressed in LNCaP-derived cells, 22Rv1 cells harbor an AR with an in-frame duplication of exon 3, resulting in an extra second zinc finger in the AR DBD (Tepper et al., Cancer Res, 62:6606-6614 (2002)). Similar to C4-2 cells, the 22Rv1 cells displayed constitutively high PSA mRNA expression that resulted from ligand- and AF-2-independent AR activity (FIG. 15). Indeed, AR-targeted siRNA inhibited androgen-independent MMTV activity in 22Rv1 cells (FIG. 8D). Moreover, similar to C4-2 cells, hARsr TAU5 deletion impaired androgen-independent MMTV activity approximately 50% (FIG. 8D). Together, these findings demonstrate that TAU5 plays a selective role in mediating ligand-independent AR activity in ADI prostate cancer cells.

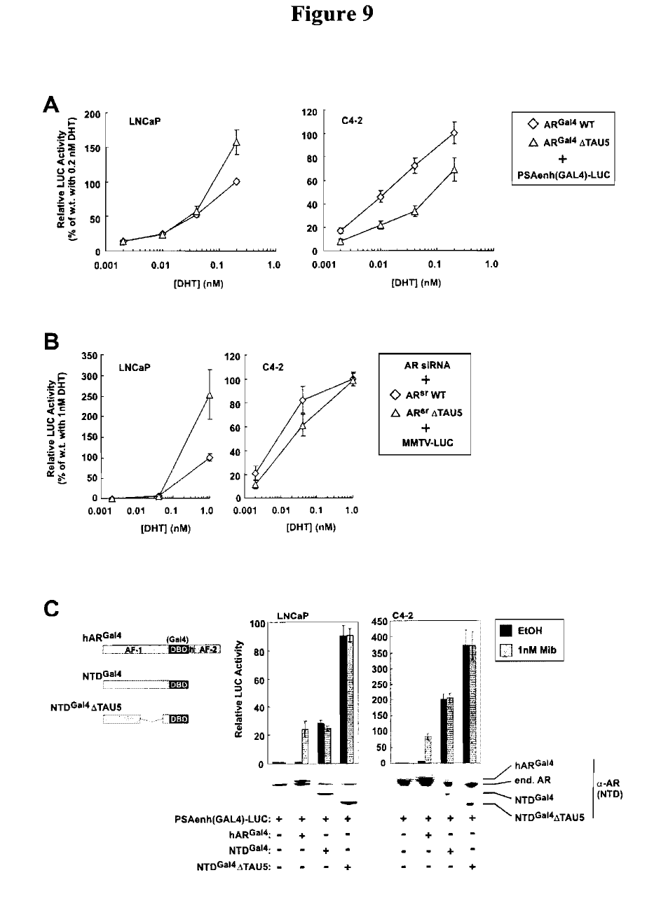

While a complete androgen-depleted environment can be achieved in vitro, such a situation may not arise in vivo. For example, data from a rapid autopsy study demonstrated that, despite androgen ablation, intraprostatic androgen levels in prostate cancer patients persist at levels sufficient to weakly transactivate the AR (Mohler et al., Clin Cancer Res, 10:440-448 (2004)). Therefore, the role of TAU5 in the AR transcriptional response to castrate-levels of its natural ligand, DHT, was explored. In LNCaP cells, the transcriptional activity of hAR^{Gal4} ΔTAU5 was higher than hAR^{Gal4} at DHT concentrations higher than 0.1 nM (FIG. 9A). Conversely, in C4-2 cells, hAR^{Gal4} ΔTAU5 transcriptional activity was approximately 50% lower than wild-type hAR^{Gal4} transcriptional activity over all DHT concentrations studied (FIG. 9A). When these experiments were performed using an AR replacement strategy and MMTV-LUC as a reporter, a similar relationship was observed (FIG. 9B). In LNCaP cells, ΔTAU5 hAR^{sr} activity was higher than wild-type hARsr at DHT concentrations higher than 0.1 nM (FIG. 9B). However, in C4-2 cells, hARsr displayed higher activity than hARsr ΔTAU5 at DHT concentrations lower than 10 pM (FIG. 9B). At concentrations higher than 0.1 nM DHT, TAU5 activity was not apparent in C4-2 cells (FIG. 9B). Together, these results suggest that TAU5 plays an important role in mediating AR activation, only under conditions of no/low androgens in ADI prostate cancer cells.

A consistent observation was that treatment of C4-2 cells with 1 nM androgens abolished TAU5 activity in C4-2 cells (FIGS. 8B, 9B, and 14), suggesting that full AR activity does not require TAU5. Deletion of the AR C-terminal domain (CTD) results in full AR activation through relieved inhibition of the AR NTD (Dehm and Tindall, J Biol Chem, 281:27882-27893 (2006); Jenster et al., J Biol Chem, 270:7341-7346 (1995); Callewaert et al., Cancer Res., 66:543-553 (2006); Chamberlain et al., J Biol Chem, 271:26772-26778 (1996)). The effect of TAU5 deletion was assessed in the context of a truncated version of hAR^{Gal4} (NTD^{Gal4}; FIG. 9C). In both LNCaP and C4-2 cells, NTD^{Gal4} was constitutively active, and did not respond to androgens (FIG. 9C). Deletion of TAU5 further increased NTD^{Gal4} activity in both cell lines, confirming that full AR activation does not require TAU5 activity (FIG. 9C). Together, these data demonstrate that TAU5 is inactive or inhibitory when the AR is fully activated.

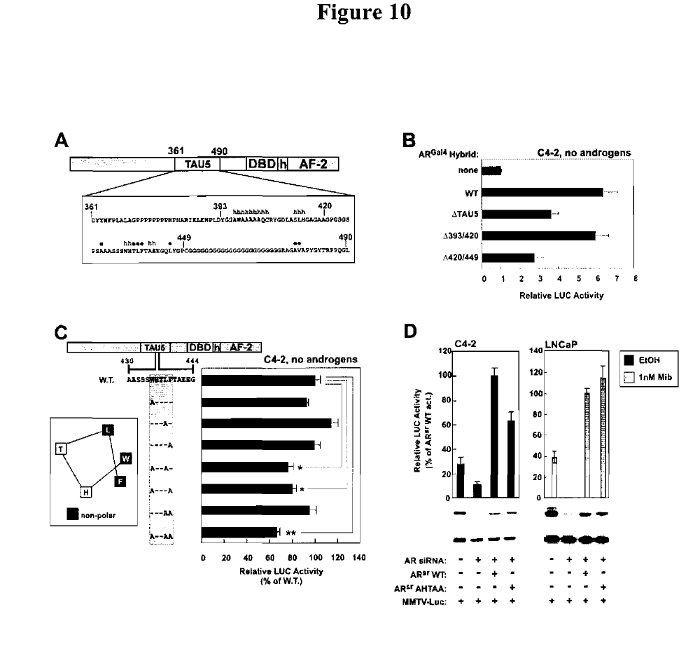

Results presented herein suggest the existence of a transactivation domain within AR TAU5 that could account for approximately 50% of the ligand-independent and/or ligand-hypersensitive AR activity in ADI prostate cancer cells. It was hypothesized that patches of rigid secondary structure within this highly disordered domain could serve as important protein interaction sites. The neural network nnpredict secondary structure prediction algorithm (Kneller et al., J Mol Biol, 214:171-182 (1990)) was applied to the amino acid sequence representing AR TAU5. Two potential sites of extended secondary structure were identified (FIG. 10A). To test whether either of these regions of putative secondary structure was important for ligand-independent AR activity in C4-2 cells, each was independently deleted from hAR^{Gal4}. Deletion of amino acids 420-449, but not amino acids 393-420, inhibited ligand-independent hAR^{Gal4} transcriptional activity to a similar degree as deletion of the entire TAU5 domain (FIG. 10B). Within the 420-449 region, a span of predicted secondary structure encompassed ⁴³⁵WHTLF⁴³⁹ (SEQ ID NO:9; FIG. 10A), which is described as a short helical motif that can mediate androgen-dependent interaction with AF-2, the C-terminal AR coactivator binding surface (He et al., J Biol Chem, 275:22986-22994 (2000); He et al., J Biol Chem, 277:25631-25639; Epub 22002 May 25638 (2002); Hur et al., PLoS Biol, 2:E274 (2004)). X-ray crystallography studies have shown that peptides containing a core WxxLF sequence adopt a helical conformation (Hur et al., PLoS Biol, 2:E274 (2004)), which, as demonstrated by a helical wheel (FIG. 10C, inset), would result in a hydrophobic surface created by the non-polar side chains of W435, LA38, and F439. To test the potential role of these hydrophobic residues within AR ⁴³⁵WHTLF⁴³⁹ (SEQ ID NO:9) in regulating ligand-independent AR transcriptional activity in ADI C4-2 cells, the hAR^{Gal4} based tethering system was employed (FIG. 10C). When the non-polar W435, L438, and F439 residues were individually substituted with alanine, no effect on androgen-independent hAR^{Gal4} transcriptional activity was observed (FIG. 10C). However, a W435A/L438A/F439A compound mutation, which eliminated all bulky hydrophobic side chains within ⁴³⁵WHTLF⁴³⁹ (SEQ ID NO:9), elicited about a 40% drop in androgen-independent hAR^{Gal4} transcriptional activity (FIG. 10C). These results suggest that AR ⁴³⁵WHTLF⁴³⁹ (SEQ ID NO:9) could mediate selective TAU5 activity in ADI prostate cancer cells. To further test this possibility, the ligand-dependent and ligand-independent activities of wild-type and AHTAA (SEQ ID NO:3) mutant hAR^{sr} were assessed in AR replacement experiments. Whereas hAR^{sr} AHTAA (SEQ ID NO:3) exhibited reduced androgen-independent activity in C4-2 cells, there was no effect on ligand-dependent activity in LNCaP cells (FIG. 10D).

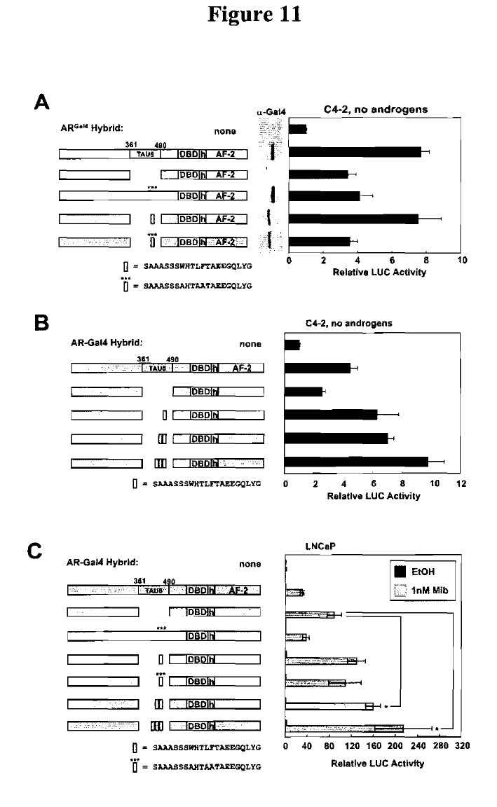

To directly assess the relationship between TAU5 and ⁴³⁵WHTLF⁴³⁹ (SEQ ID NO:9), the transcriptional activities of ΔTAU5 and AHTAA (SEQ ID NO:3) versions of hAR^{Gal4} were compared using the promoter tethering assay in C4-2 cells (FIG. 11A). Both TAU5 deletion and AHTAA (SEQ ID NO:3) mutation impaired hAR^{Gal4} ligand-independent transcriptional activity approximately 50% (FIG. 11A). Importantly, a 21-amino acid peptide containing the core ⁴³⁵WHTLF⁴³⁹ (SEQ ID NO:9), but not AHTAA (SEQ ID NO:3), rescued full ligand-independent transcriptional activity of hAR^{Gal4} ΔTAU5 in C4-2 cells (FIG. 11A). These results demonstrate that the AR ⁴³⁵WHTLF⁴³⁹ (SEQ ID NO:9) motif plays a direct role in mediating TAU5 activity in ADI C4-2 cells. These results also indicate that NTD ⁴³⁵WHTLF⁴³⁹ (SEQ ID NO:9) is an important TAU5 motif that selectively mediates ligand-independent AR activity.

Results presented herein suggest that AR WHTLF (SEQ ID NO:9) could represent a novel N-terminal AR transactivation domain. Aberrant AR activation could also be facilitated by stabilization of the AR in the absence of ligand via an aberrant N/C interaction between ⁴³⁵WHTLF⁴³⁹ (SEQ ID NO:9) and AF-2. Indeed, structural and functional studies have demonstrated that ligand-bound AR can be stabilized by an interaction between the AR AF-2 domain and ²³FQNLF²⁷ (SEQ ID NO:22) or ⁴³⁵WHTLF⁴³⁹ (SEQ ID NO:9) motifs in the AR NTD (He et al., J Biol Chem, 275:22986-22994 (2000); Hur et al., PLoS Biol, 2:E274 (2004); Estebanez-Perpina et al., J Biol Chem, 280:8060-8068; Epub November 2004 8024 (2005); Ikonen et al., J Biol Chem, 272:29821-29828 (1997); He et al., J Biol Chem, 274:37219-37225 (1999); He et al., J Biol Chem, 276:42293-42301; Epub 42001 September 42210 (2001)). To differentiate between these scenarios, one, two, or three ⁴³⁵WHTLF⁴³⁹ (SEQ ID NO:9)-containing peptides were added to hAR^{Gal4} ΔTAU5. Stepwise increases in ligand-independent hAR^{Gal4} transcriptional activity were observed in C4-2 cells with each ⁴³⁵WHTLF⁴³⁹ (SEQ ID NO:9)-containing peptide inserted (FIG. 11B). When similar experiments were performed in LNCAP cells, it was surprising to observe that insertion of two or three ⁴³⁵WHTLF⁴³⁹ (SEQ ID NO:9)-containing peptides in hAR^{Gal4} ΔTAU5 resulted in ligand-dependent super-activation of the AR (FIG. 11C).

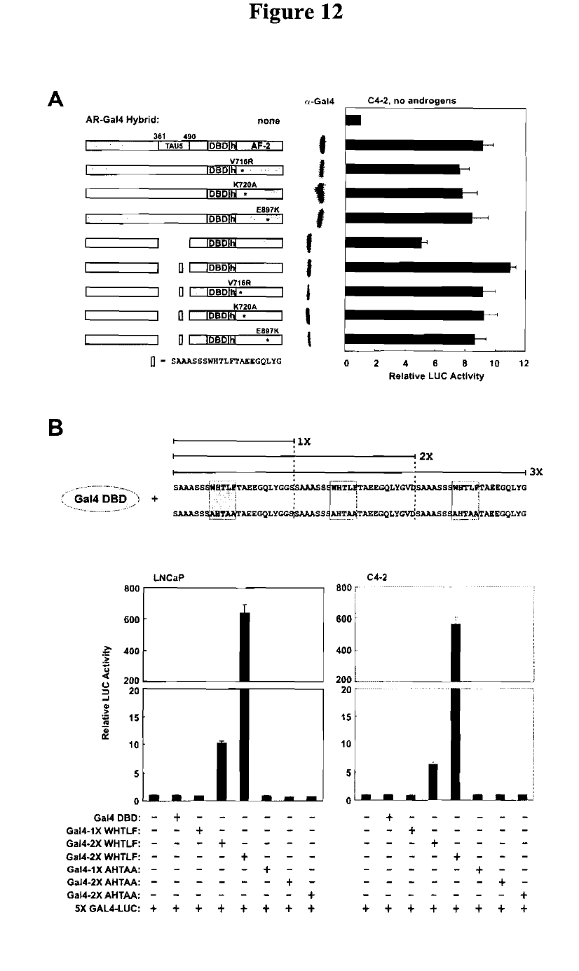

While these results were consistent with a transactivation role for ⁴³⁵WHTLF⁴³⁹ (SEQ ID NO:9), they did not rule out the possibility that multiple copies of ⁴³⁵WHTLF⁴³⁹ (SEQ ID NO:9) were mediating more efficient interactions with AF-2, resulting in stronger N/C stabilization and subsequent higher levels of AR activity. AF-2 function was therefore abolished by incorporating three separate mutations in V716, K720, and E897, which are AR residues that comprise the conserved nuclear receptor AF-2 charge-clamp and mediate binding with AR ⁴³⁵WHTLF⁴³⁹ (SEQ ID NO:9; He et al., J Biol Chem, 277:25631-25639; Epub 22002 May 25638 (2002); Hur et al., PLoS Biol, 2:E274 (2004)). Mutations in these charge-clamp residues impair ligand-dependent AR transcriptional activity in LNCaP as well as other cell lines (Dehm and Tindall, J Biol Chem, 281:27882-27893 (2006); He et al., J Biol Chem, 277:25631-25639; Epub 22002 May 25638 (2002); Estebanez-Perpina et al., J Biol Chem, 280:8060-8068; Epub November 2004 8024 (2005); He et al., J Biol Chem, 274:37219-37225 (1999); Alen et al., Mol Cell Biol., 19:6085-6097 (1999)). However, in C4-2 cells, mutations in these charge-clamp residues did not disrupt the ligand-independent activity of wild-type hAR^{Gal4}, nor hAR^{Gal4} ΔTAU5 with a 21-amino acid insert harboring the AR ⁴³⁵WHTLF⁴³⁹ (SEQ ID NO:9) motif (FIG. 12A). These data argue against a role for ⁴³⁵WHTLF⁴³⁹ (SEQ ID NO:9) in mediating an aberrant N/C interaction in ADI prostate cancer cells, and indicate that ⁴³⁵WHTLF⁴³⁹ (SEQ ID NO:9) represents an AR transactivation domain.

To directly assess a role for the AR ⁴³⁵WHTLF⁴³⁹ (SEQ ID NO:9) motif in transcriptional activation, one, two, or three copies of an AR-derived peptide with a core ⁴³⁵WHTLF⁴³⁹ (SEQ ID NO:9) sequence were tethered to the Gal4 DBD and tested for their ability to stimulate transcription from a GAL4-regulated luciferase reporter. In both LNCaP and C4-2 cells, a Gal4 DBD containing two ⁴³⁵WHTLF⁴³⁹ (SEQ ID NO:9) peptides mediated transcriptional activation (FIG. 12B). Of particular note, three tandem copies of ⁴³⁵WHTLF⁴³⁹ (SEQ ID NO:9) peptides activated transcription of the GAL4-driven promoter 600-fold in both cell lines. These results, together with the results presented above, further indicate that the AR ⁴³⁵WHTLF⁴³⁹ (SEQ ID NO:9) motif is a transcriptional activation domain. Although this motif has the potential to stimulate transcription in both LNCaP and C4-2 cells (FIGS. 11C and 12B), results presented herein indicate that in the context of full-length AR, ⁴³⁵WHTLF⁴³⁹ (SEQ ID NO:9) selectively mediates transcriptional activation under no/low androgen conditions in ADI prostate cancer cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a molecule capable of inhibiting or activating androgen-independent androgen receptor polypeptide activity, said method comprising determining whether or not a test molecule interacts with said androgen receptor polypeptide in a manner requiring one or more amino acids residues of said androgen receptor polypeptide selected from the group consisting of amino acids residues 181, 182, 435, 438, 439, 101-211, 253-361, and 361-490 of the sequence set forth in SEQ ID NO: 1, wherein the presence of said interaction indicates that said test molecule is said molecule.

2. A method of identifying a molecule capable of inhibiting or activating androgen receptor polypeptide activity, said method comprising:
(a) contacting cells with a test molecule, wherein said cells comprise:
a first nucleic acid comprising a nucleotide sequence that encodes (i) an androgen receptor polypeptide or fragment thereof and (ii) a DNA binding domain, and
a second nucleic acid comprising a nucleotide sequence that encodes a reporter gene operably linked to one or more recognition sequences for said DNA binding domain, and
(b) determining whether or not the expression level of said reporter gene is decreased or increased compared to the level of expression of said reporter gene in control cells not contacted with said test molecule,
wherein a decrease in said expression of said reporter gene indicates that said test molecule is a molecule capable of inhibiting androgen receptor polypeptide activity, and wherein an increase in said expression of said reporter gene indicates that said test molecule is a molecule capable of activating androgen receptor polypeptide activity.

3. The method of claim 2, wherein said cells are androgen-independent prostate cancer cells.

4. The method of claim 2, wherein said test molecule is a polypeptide.

5. The method of claim 2, wherein said test molecule is an antibody.

6. The method of claim 2, wherein said test molecule is a small molecule.

7. The method of claim 2, wherein said first nucleic acid comprises a nucleotide sequence that encodes a human androgen receptor polypeptide lacking a DNA binding domain.

8. The method of claim 2, wherein said first nucleic acid comprises a nucleotide sequence that encodes a Gal4 DNA binding domain.

9. An isolated nucleic acid molecule comprising a nucleotide sequence encoding a human androgen receptor polypeptide comprising the amino acid sequence set forth in SEQ ID NO: 1 with the proviso that said amino acid sequence comprises a mutation at an amino acid residue selected from the group consisting of amino acid residues 435, 438, and 439.

10. The isolated nucleic acid molecule of claim 9, wherein said amino acid sequence comprises a substitution at amino acid residue 435.

11. The isolated nucleic acid molecule of claim 9, wherein said amino acid sequence comprises a deletion at amino acid residue 435.

12. The isolated nucleic acid molecule of claim 9, wherein said amino acid sequence comprises an insertion at amino acid residue 435.

13. An isolated nucleic acid molecule comprising a nucleotide sequence encoding a human androgen receptor polypeptide comprising the amino acid sequence set forth in SEQ ID NO:1 with the proviso that said amino acid sequence lacks the amino acid sequence at residues 101-211, 253-361, or 361-490.


SEQUENCE LISTING (46 sequences)

SEQ ID NO: 1 - protein, 919 aa, Homo sapiens
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - protein, 5 aa, Artificial Sequence
  artificial sequence based on androgen receptor motif
AQNAA

SEQ ID NO: 3 - protein, 5 aa, Artificial Sequence
  artificial sequence based on androgen receptor motif
AHTAA

SEQ ID NO: 4 - protein, 28 aa, Homo sapiens
PSTLSLLGPTFPGLSSCSADIKDILSEA

SEQ ID NO: 5 - protein, 6 aa, Artificial Sequence
  artificial sequence based on androgen receptor motif
PNTLNL

SEQ ID NO: 6 - protein, 8 aa, Artificial Sequence
  artificial sequence based on androgen receptor motif
IKDNNSEA

SEQ ID NO: 7 - protein, 16 aa, Homo sapiens
GTLELPSTLSLYKSGA

SEQ ID NO: 8 - protein, 15 aa, Homo sapiens
AASSSWHTLFTAEEG

SEQ ID NO: 9 - protein, 5 aa, Homo sapiens
WHTLF

SEQ ID NO: 10 - protein, 8 aa, Homo sapiens
IKDILSEA

SEQ ID NO: 11 - protein, 128 aa, Homo sapiens
DYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGL

SEQ ID NO: 12 - protein, 21 aa, Homo sapiens
SAAASSSWHTLFTAEEGQLYG

SEQ ID NO: 13 - protein, 21 aa, Artificial Sequence
  artificial sequence based on androgen receptor motif
SAAASSSAHTAATAEEGQLYG

SEQ ID NO: 14 - protein, 67 aa, Artificial Sequence
  artificial sequence based on androgen receptor motif
SAAASSSWHTLFTAEEGQLYGGSSAAASSSWHTLFTAEEGQLYGVDSAAASSSWHTLFTAEEGQLYG

SEQ ID NO: 15 - protein, 67 aa, Artificial Sequence
  artificial sequence based on androgen receptor motif
SAAASSSAHTAATAEEGQLYGGSSAAASSSAHTAATAEEGQLYGVDSAAASSSAHTAATAEEGQLYG

SEQ ID NO: 16 - DNA, 38 bp, Artificial Sequence
  oligonucleotide
ccaccccagaagacgcgtctgatctgtggagatgaagc

SEQ ID NO: 17 - DNA, 38 bp, Artificial Sequence
  oligonucleotide
gcttcatctccacagatcagacgcgtcttctggggtgg

SEQ ID NO: 18 - DNA, 38 bp, Artificial Sequence
  oligonucleotide
gggatgactctgggaccgcggaagctgaagaaacttgg

SEQ ID NO: 19 - DNA, 38 bp, Artificial Sequence
  oligonucleotide
ccaagtttcttcagcttccgcggtcccagagtcatccc

SEQ ID NO: 20 - DNA, 22 bp, Artificial Sequence
  oligonucleotide
gaaagacgcgtctactgtcttc

SEQ ID NO: 21 - DNA, 21 bp, Artificial Sequence
  oligonucleotide
aattcccgcggtacagtcaac

SEQ ID NO: 22 - protein, 5 aa, Homo sapiens
FQNLF

SEQ ID NO: 23 - protein, 6 aa, Homo sapiens
PSTLSL

SEQ ID NO: 24 - protein, 5 aa, Artificial Sequence
  artificial sequence based on androgen receptor motif
WHTAA

SEQ ID NO: 25 - protein, 5 aa, Artificial Sequence
  artificial sequence based on androgen receptor motif
AHTAF

SEQ ID NO: 26 - protein, 5 aa, Artificial Sequence
  artificial sequence based on androgen receptor motif
AHTLA

SEQ ID NO: 27 - RNA, 21 nt, Artificial Sequence
  siRNA oligonucleotide
caagggagguuacaccaaauu

SEQ ID NO: 28 - RNA, 21 nt, Artificial Sequence
  siRNA oligonucleotide
gaaaugauugcacuauugauu

SEQ ID NO: 29 - DNA, 47 bp, Artificial Sequence
  oligonucleotide
cgaggcagctgcgtaccaggcgcgcgactactacaactttccactgg

SEQ ID NO: 30 - DNA, 47 bp, Artificial Sequence
  oligonucleotide
ccagtggaaagttgtagtagtcgcgcgcctggtacgcagctgcctcg

SEQ ID NO: 31 - DNA, 42 bp, Artificial Sequence
  oligonucleotide
cggccccctcaggggctggcgcgccaggaaagcgacttcacc

SEQ ID NO: 32 - DNA, 38 bp, Artificial Sequence
  oligonucleotide
ctggagaacccgctggcgcgcggcagcgcctgggcggc

SEQ ID NO: 33 - DNA, 38 bp, Artificial Sequence; identical to SEQ ID NO: 32
  oligonucleotide

SEQ ID NO: 34 - DNA, 38 bp, Artificial Sequence
  oligonucleotide
gccgcccaggcgctgccgcgcgccagcgggttctccag

SEQ ID NO: 35 - DNA, 37 bp, Artificial Sequence
  oligonucleotide
catggcgcgggtgcagcgcgccccggttctgggtcac

SEQ ID NO: 36 - DNA, 37 bp, Artificial Sequence
  oligonucleotide
gtgacccagaaccggggcgcgctgcacccgcgccatg

SEQ ID NO: 37 - DNA, 38 bp, Artificial Sequence
  oligonucleotide
ggccagttgtatggagcgcgcggtggtggtgggggtgg

SEQ ID NO: 38 - DNA, 38 bp, Artificial Sequence
  oligonucleotide
ccacccccaccaccaccgcgcgctccatacaactggcc

SEQ ID NO: 39 - DNA, 69 bp, Artificial Sequence
  oligonucleotide
cgcgctcagccgccgcttcctcatcctggcacactctcttcacagccgaagaaggccagttgtatggag

SEQ ID NO: 40 - DNA, 69 bp, Artificial Sequence
  oligonucleotide
cgcgctccatacaactggccttcttcggctgtgaagagagtgtgccaggatgaggaagcggcggctgag

SEQ ID NO: 41 - DNA, 132 bp, Artificial Sequence
  oligonucleotide
cgcgctcagccgccgcttcctcatcctggcacactctcttcacagccgaagaaggccagttgtatggatcagccgccgcttcctcatcctggcacactctcttcacagccgaagaaggccagttgtatggag

SEQ ID NO: 42 - DNA, 132 bp, Artificial Sequence
  oligonucleotide
cgcgctccatacaactggccttcttcggctgtgaagagagtgtgccaggatgaggaagcggcggctgatccatacaactggccttcttcggctgtgaagagagtgtgccaggatgaggaagcggcggctgag

SEQ ID NO: 43 - DNA, 195 bp, Artificial Sequence
  oligonucleotide
cgcgctcagccgccgcttcctcatcctggcacactctcttcacagccgaagaaggccagttgtatggatcagccgccgcttcctcatcctggcacactctcttcacagccgaagaaggccagttgtatggatcagccgccgcttcctcatcctggcacactctcttcacagccgaagaaggccagttgtatggag

SEQ ID NO: 44 - DNA, 195 bp, Artificial Sequence
  oligonucleotide
cgcgctccatacaactggccttcttcggctgtgaagagagtgtgccaggatgaggaagcggcggctgatccatacaactggccttcttcggctgtgaagagagtgtgccaggatgaggaagcggcggctgatccatacaactggccttcttcggctgtgaagagagtgtgccaggatgaggaagcggcggctgag

SEQ ID NO: 45 - DNA, 69 bp, Artificial Sequence
  oligonucleotide
cgcgctcagccgccgcttcctcatccgcgcacactgccgccacagccgaagaaggccagttgtatggag

SEQ ID NO: 46 - DNA, 69 bp, Artificial Sequence
  oligonucleotide
cgcgctccatacaactggccttcttcggctgtggcggcagtgtgcgcggatgaggaagcggcggctgag